(19) United States
(12) Patent Application Publication
(10) Pub. No.: US 2026/0117159 A1
(43) Pub. Date: Apr. 30, 2026

(54) Cell Suspension Culture Device Provided With Flow Diverter

(71) Applicant: Cellex S.r.l., Rome (IT)

(72) Inventors: Federica Liguori, Rome (IT); Margherita Scamarcio, Rome (IT); Michele Pistillo, Rome (IT); Maurizio Mastantuono, Rome (IT); Giacomo Di Benedetto, Rome (IT); Giuseppe Falvo D'Urso Labate, Rome (IT)

(21) Appl. No.: 19/163,873

(22) PCT Filed: Mar. 12, 2024
(86) PCT No.: PCT/IB2024/052367

§ 371 Date: Sep. 10, 2025

(30) Foreign Application Priority Data
Mar. 15, 2023 (IT) 102023000004875

(51) Int. Cl. C12M 1/00; C12M 3/00

(52) U.S. Cl. CPC C12M 27/18; C12M 21/08; C12M 23/34; C12M 23/40; C12M 23/48; C12M 29/00

(57) ABSTRACT

The present invention relates to a cell suspension culture device (1) comprising: a culture chamber (2), a fluid inlet portion (11) configured and a fluid outlet portion (14). The device (1) further comprising a flow diverter (21) disposed within the culture chamber (2) and, with reference to a fluid feed direction, between the fluid inlet portion (11) and the fluid outlet portion (14). The flow diverter (21) has: at least one fluid inlet portion (22) in fluid communication with the fluid inlet portion (1) and has a plurality of fluid admittance outlets (25) in fluid communication with the fluid inlet portion (22). The flow diverter (21) is configured to input, via the plurality of fluid admittance outlets (25), a plurality of fluid flows into the culture chamber (2). In an embodiment, the device (1) comprises at least one scaffold holder (50).

### TECHNICAL FIELD OF THE INVENTION

The present invention relates to a cell suspension culture device provided with a flow diverter. The culture device can be configured to allow both suspension and adhesion culture.

The invention also relates to a method of operating a cell suspension culture device, a use of the device and a process for assembling a culture device.

### PRIOR ART

Cell culture devices, also known as microgravity-generating devices, in which cells are grown in suspension from a moving fluid within a flow chamber, or culture chamber, are known.

Such devices can range in size from a few millilitres to several tens of litres, and are used in the laboratory to perform tests on cells grown in suspension, or in the industrial environment for large-scale production of cells for scientific or industrial use, or in the vaccine production chain.

In known devices, suspension is created either actively or passively. In ‘active’ devices, suspension is created by means of rotating components set in motion by dedicated actuators. In ‘passive’ devices, on the other hand, there are no such rotating components.

It is also to be said that all these devices, whether ‘active’ or ‘passive’, do not ensure proper diffusion of oxygen and nutrients at the region of the motion field where the cells are suspended. This results in low rates of cell proliferation and production, rates that can, moreover, only be assessed empirically.

International patent application WO2020095143A1, in the name of the same Applicant, sought to improve passive devices with respect to the handling of the flow entering the culture chamber. This improvement involves the provision of a valve organ moved by a mechanical or magnetic handling device.

The Applicant has noted that the device of WO2020095143A1, while having considerable advantages over the prior art, can be improved in some respects. In particular, said device has room for improvement in terms of handling of the cells and/or of particulates in suspension as the cells tended to accumulate and to adhere in the lower part of the culture chamber.

### AIMS OF THE INVENTION

The main purpose of the present invention is therefore to overcome the drawbacks previously described in connection with the prior art.

The aim of the invention is to improve the handling of cells and/or particulates in a cell suspension culture device.

Going into greater detail, the aim of the invention is to improve the handling of cells and/or particulates in suspension by optimising the motion field generated within the culture chamber.

It is therefore the purpose of the present invention to provide a cell suspension culture device that allows optimal growth of the cells in the culture chamber due to the motion field generated within the culture chamber.

These and other purposes are achieved by a flow diverter, a cell suspension culture device equipped with a flow diverter and a method of operation of a cell culture device in accordance with the following description, the appended claims and the following aspects.

### SUMMARY OF THE INVENTION

Aspects of the invention, which form an integral part of the technical content of this patent text, are described herein. These aspects may be used to limit claims and/or define further claims during the life of this patent right.

The Applicant noted that the above-mentioned purposes are achieved by the provision of a flow diverter that can be placed within the culture chamber of a cell suspension culture device, in particular at a lower portion of the culture chamber.

This flow diverter changes the flow lines within the chamber in such a way that at least one toroidal and/or one rotational motion field are/is generated.

By means of a computational and experimental study, the Applicant has noticed and verified that such a flow diverter, when placed inside a culture chamber of a cell suspension culture device, enables to:
reduce cell adhesion on the wall of the culture device;
more easily control the shear stresses experienced by the cells so that they can be modified at will according to the type of cells being cultured;
increase the homogenisation of nutrients and oxygen and avoid the formation of gradients of concentration within the culture volume.

Numbered aspects of the invention follow.
1. Flow diverter comprising:
   at least one fluid inlet, and
   at least one fluid admittance outlet section or a plurality of fluid admittance outlets, in fluid communication with said at least one fluid inlet,
the flow diverter being configured and intended to co-operate with a cell culture device, the device comprising:
   a culture chamber,
   at least one fluid inlet portion configured to allow fluid to enter the culture chamber,
   at least one fluid outlet portion configured to allow fluid output from the culture chamber, preferably the flow diverter, in a coupling configuration where it is coupled to the cell culture device, being configured for:
      conveying fluid from said at least a fluid inlet portion into the culture chamber,
      admit, by means of said fluid admittance section or plurality of fluid admittance outlets, at least one fluid flow or a plurality of fluid flows into the culture chamber.
2. Aspect according to aspect 1, in which the flow diverter, in a coupling configuration in which it is coupled to the cell culture device, is configured to generate turbulence in a fluid.
3. Aspect according to aspect 1 or 2, in which the flow diverter is designed and intended for use inside the culture chamber.
4. Aspect according to aspect 1 or 2 or 3, wherein the flow diverter is intended to be used in cooperation with a cell suspension culture device.
5. Use of the flow diverter in accordance with any of the preceding aspects to admit a plurality of fluid flows into the culture chamber of a cell culture device, preferably cell suspension culture device.
6. Use according to aspect 5, wherein the use involves generating at least one toroidal motion of fluid and/or one rotational motion of fluid in the culture chamber.
7. Cell culture device, preferably cell suspension culture device, comprising:
   a culture chamber,
   at least one fluid inlet portion configured to enable fluid destined to the culture chamber to enter,
   at least one fluid outlet portion configured to enable exit of fluid from the culture chamber,
   a flow diverter arranged at least partially inside the chamber and arranged, with reference to a fluid advancement direction, between said at least one fluid inlet portion and said at least one fluid outlet portion,
the flow diverter having:
   at least one fluid inlet in fluid communication with said at least one fluid inlet portion, and
   at least one fluid admittance outlet section or a plurality of fluid admittance outlets in fluid communication with said at least one fluid inlet of the flow diverter,
   preferably the flow diverter being configured to:
      convey into the culture chamber fluid from the said at least a portion of the fluid inlet portion,
      admit, through said fluid admittance outlet section or plurality of fluid admittance outlets, a fluid flow or a plurality of fluid flows into the culture chamber.
8. Aspect according to any of the above aspects, in which the flow diverter is monolithic, preferably made by 3D printing.
9. Aspect according to any of the previous aspects, in which the fluid admittance outlets are defined on a side and/or bottom surface of the flow diverter.
10. Aspect according to any one of the preceding aspects, wherein each fluid admittance outlet of the plurality of fluid admittance outlets is directed transversely to a wall of the culture chamber, in particular transversely to a side wall and/or a bottom wall of the culture chamber.
11. Aspect according to any one of the preceding aspects, wherein each fluid admittance outlet of the plurality of fluid admittance outlets has an axis defining a fluid outlet direction, each axis being transverse to a wall of the culture chamber, in particular transverse to a bottom wall and/or a side wall of the culture chamber.
12. Aspect according to any one of the preceding aspects, wherein the flow diverter comprises at least one stem provided with a fluid supply conduit and further comprises a body in fluid communication with said fluid supply conduit, said fluid supply conduit being in fluid communication, in particular in direct fluid communication (i.e., without intermediate components), with said at least one fluid inlet portion and being configured to convey a fluid flow to said body.
13. Aspect according to any of the previous aspects, in which the fluid supply conduit is an internal conduit, i.e. it is cut inside a body of the stem of the flow diverter.
14. Aspect according to any of the preceding aspects, in which the body has a bottom portion defining a concavity developing annularly around the stem.
15. Aspect according to any of the preceding aspects, in which said concavity of the bottom portion of the stem corresponds to a convexity, in particular defined by a convex edge, of a bottom wall of the chamber.
16. Aspect according to any of the preceding aspects, in which said at least one fluid supply conduit is arranged at a central portion of the flow diverter.
17. Aspect according to any of the preceding aspects, wherein the body develops inside the culture chamber.
18. Aspect according to any of the previous aspects, in which the body has a greater volume than the stem.
19. Aspect according to any of the previous aspects, in which the body is tapered from the bottom upwards.
20. Aspect according to any of the preceding aspects, in which the body of the flow diverter comprises a perimeter portion, said plurality of fluid admittance openings being defined at the perimeter portion.
21. Aspect according to any of the previous aspects, where the perimeter portion is a circumferential portion of the body of the flow diverter.
22. Aspect according to any of the preceding aspects, in which the outlets of the plurality of fluid admittance outlets are angularly offset from each other.
23. Aspect according to any one of the preceding aspects, in which the outlets of the plurality of fluid admittance outlets are angularly offset from each other with reference to a circumferential direction.
24. Aspect according to any of the preceding aspects, in which the outlets of the plurality of fluid admittance outlets are angularly offset from each other by the same angle, the offset angle being defined between consecutive fluid admittance outlets.
25. Aspect according to any one of the preceding aspects, wherein the flow diverter has an annular conduit arranged between said at least one fluid inlet and said plurality of fluid admittance outlets, the outlets of the plurality of fluid admittance outlets being angularly staggered along said annular conduit.
26. Aspect according to any of the preceding aspects, in which the outlets of the plurality of fluid admittance outlets have radial development.
27. Aspect according to any of the preceding aspects, in which the supply conduit develops at a lower portion of the flow diverter, the body developing at an upper portion of the flow diverter.
28. Aspect according to any one of the preceding aspects, wherein the fluid supply conduit defines said at least one fluid inlet and is configured to convey fluid to the plurality of fluid admittance outlets.
29. Aspect according to any of the previous aspects, in which the flow diverter has a double function:
   conveying fluid from said at least one fluid inlet portion into the culture chamber so as to divide it, in particular equally divide it (i.e. to divide it into flows with corresponding or similar flow rates), into a plurality of fluid flows that are fed, via said plurality of fluid admittance outlets, into the culture chamber,
   let fluid coming from the culture chamber flow outside the culture chamber.
30. Aspect according to any of the preceding aspects, in which the flow diverter is a component constituting a fluid handling interface configured to cooperate with the culture chamber in order to allow fluid to flow into and out of the culture chamber.
31. Aspect according to any of the previous aspects, wherein:
   the flow diverter also includes at least one fluid outflow outlet to allow fluid from the culture chamber to flow out of the chamber, optionally, the fluid outflow outlet is in communication with said at least one fluid outlet portion.
32. Aspect according to any one of the preceding aspects, wherein the flow diverter is configured to convey fluid from said culture chamber, via said fluid outflow outlet, to said at least one fluid outlet portion so as to evacuate fluid from the device.
33. Aspect according to any of the above aspects, in which the flow diverter is at least partially arranged at a lower portion of the chamber.
34. Aspect according to any of the preceding aspects, in which said at least one output portion is arranged at a lower portion of the cell suspension culture device.
35. Aspect according to any one of the preceding aspects, wherein the flow diverter comprises a fluid outflow conduit connecting said at least one fluid outflow outlet with said at least one fluid outlet portion.
36. Aspect according to any of the previous aspects, wherein:
   the fluid outflow conduit runs between a first end and a second end, the first end being defined at an upper portion, e.g. a top portion, of the flow diverter,
   said at least one fluid outflow outlet is defined at the second end.
37. Aspect according to any of the preceding aspects, in which the first end is configured to channel fluid from the culture chamber into the outflow conduit.
38. Aspect according to any of the preceding aspects, in which the fluid outflow conduit is formed at least partially inside the body.
39. Aspect according to any of the previous aspects, in which the flow diverter is engaged at a lower portion of the device.
40. Aspect according to any of the above aspects, in which the device has a lower portion that is defined below the bottom wall of the culture chamber.
41. Aspect according to any of the previous aspects, in which the stem of the flow diverter is engaged at a housing of the device that is defined at a lower portion of the device.
42. Aspect according to any one of the preceding aspects, wherein the flow diverter comprises a fluid outflow conduit extending between a first end and a second end, the first end being defined at an upper portion, e.g. a top portion, of the flow diverter, said at least one fluid outflow outlet being defined at the second end.
43. Aspect according to any one of the preceding aspects, wherein the flow diverter comprises at least one fluid distribution conduit defined in the body and arranged between said at least one fluid supply conduit and one or more outlets of said plurality of fluid admittance outlets.
44. Aspect according to any of the previous aspects, in which the fluid distribution conduit is an inner conduit, i.e. it is formed inside a body of the flow diverter.
45. Aspect according to any one of the preceding aspects, wherein said at least one fluid distribution conduit is transverse to said at least one fluid supply conduit, having an at least partially curvilinear development and being in fluid communication with one or more fluid admittance outlets of said plurality of fluid admittance outlets.
46. Aspect according to any one of the preceding aspects, in which said at least one fluid distribution conduit has a conformation of an annulus or as an annulus portion.
47. Aspect according to any of the previous aspects, in which the fluid distribution conduit has an at least partially annular development.
48. Aspect according to any one of the preceding aspects, wherein the flow diverter comprises at least one junction conduit disposed between said at least one fluid distribution conduit and said at least one fluid supply conduit, the junction conduit being configured to convey into said at least one fluid distribution conduit the fluid from said at least one fluid supply conduit.
49. Aspect according to any of the previous aspects, in which the junction conduit has a curved conformation.
50. Aspect according to any of the preceding aspects, in which said at least one fluid supply conduit is arranged at a central portion of the flow diverter.
51. Aspect according to any one of the preceding aspects, wherein the flow diverter comprises a single fluid distribution conduit defined in the body and arranged between said at least one fluid supply conduit and said plurality of fluid admittance outlets.
52. Aspect according to any one of the preceding aspects, wherein the single fluid distribution conduit is configured to distribute fluid to all outlets of said plurality of fluid admittance outlets.
53. Aspect according to any of the preceding aspects, in which the flow diverter comprises a single fluid inlet in fluid communication with said single fluid distribution conduit.
54. Aspect according to any of the previous aspects, in which the single fluid distribution conduit has an annular shape.
55. Aspect according to any of the previous aspects, in which the single fluid distribution conduit has an annular conformation forming an open ring.
56. Aspect according to any of the previous aspects, wherein:
   the flow diverter comprises:
      a plurality of fluid inlets,
      a plurality of fluid distribution conduits, each of which is in fluid communication with a respective fluid inlet,
   in the plurality of fluid admittance outlets, a plurality of fluid admittance outlets sections or groups can be identified, each fluid admittance outlet section or group comprising at least one fluid admittance outlet and being in fluid communication with a respective fluid distribution conduit.
57. Aspect according to any one of the preceding aspects, in which the plurality of fluid admittance outlets is divided into a plurality of fluid admittance sections or groups.
58. Aspect according to any one of the preceding aspects, wherein each group of fluid admittance outlets comprises a respective plurality of fluid admittance outlets.
59. Aspect according to any one of the preceding aspects, wherein each fluid distribution conduit is in fluid communication with a group of fluid admittance outlet comprising a plurality of fluid admittance outlets.
60. Aspect according to any of the previous aspects, where each fluid admittance section comprises the same number of fluid admittance outlets.
61. Aspect according to any of the previous aspects, in which each fluid distribution conduit is fed independently of the other fluid distribution conduits.
62. Aspect according to any of the previous aspects, in which the device is configured to independently feed fluid distribution conduit, e.g. intermittently and/or sequentially.
63. Aspect according to any of the previous aspects, in which the device is configured to supply one or more fluid distribution conduits for a specific period of time.
64. Aspect according to any one of the preceding aspects, wherein said at least one fluid supply conduit is arranged at a central portion of the flow diverter, the flow diverter comprising a plurality of fluid distribution conduits branching from the fluid supply conduit to a perimeter portion of the body of the flow diverter.
65. Aspect according to any of the previous aspects, in which each fluid distribution conduit opens into a respective fluid admittance outlet.
66. Aspect according to any of the previous aspects, where the flow diverter comprises a single fluid supply conduit.
67. Aspect according to any one of the preceding aspects, in which the conduits of the plurality of fluid distribution conduits radially branch off from the single fluid supply conduit.
68. Aspect according to any of the previous aspects, in which each fluid distribution conduit has an at least partially curvilinear development.
69. Aspect according to any of the preceding aspects, in which each fluid distribution conduit runs radially away from said at least one fluid supply conduit.
70. Aspect according to any one of the preceding aspects, wherein the flow diverter has a single fluid supply conduit configured to supply fluid to said plurality of fluid supply conduits.
71. Aspect according to any of the preceding aspects, in which each fluid distribution conduit has an at least partially helicoidal development.
72. Aspect according to any of the preceding aspects, in which the device comprises an additional chamber or pre-chamber and the flow diverter is at least partially housed in said additional chamber or pre-chamber.
73. Aspect according to any of the previous aspects, in which the additional chamber or pre-chamber is arranged upstream of the culture chamber.
74. Aspect according to any of the preceding aspects, in which the fluid supply conduit is housed at said additional chamber or pre-chamber.
75. Aspect according to any of the previous aspects, wherein:
   the culture chamber comprises a bottom wall with a bottom opening and a convex edge developing around the bottom opening,
   the pre-chamber is above delimited by said bottom opening and is arranged, with reference to a fluid advancement direction, between at least a portion of the fluid inlet of the device and an internal volume of the chamber,
   The flow diverter forms, as the fluid exits the pre-chamber, a fluid passage port with the convex edge of the bottom wall of the chamber.
76. Aspect according to any of the previous aspects, in which the bottom wall of the chamber comprises a concavity around the convex edge.
77. Aspect according to any of the previous aspects, in which the convex edge is placed between the concavity and the bottom opening.
78. Aspect according to any of the preceding aspects, in which the convex edge has a convexity, the flow diverter includes, at one of its bottom walls, a concavity corresponding to the convexity of the convex edge.
79. Aspect according to any of the previous aspects, in which the fluid passage port is defined between a bottom wall of the flow diverter and a bottom wall of the culture chamber.
80. Aspect according to any of the previous aspects, wherein:
   the convex edge develops circumferentially around the bottom opening,
   the concavity develops circumferentially around the convex edge,
   the fluid passage port has an annular development.
81. Aspect according to any of the previous aspects, wherein:
   the device comprises a second fluid inlet portion in fluid communication with the pre-chamber and configured to feed fluid destined for the pre-chamber,
   the pre-chamber is arranged, with reference to a fluid advancement direction, downstream of the second fluid inlet portion and upstream of an internal volume of the culture chamber.
82. Aspect according to any of the previous aspects, wherein:
   the device comprises a plurality of fluid inlet portions,
   the flow diverter comprises a plurality of fluid distribution conduits each of which is associated with a respective fluid inlet portion and receives fluid coming from the respective fluid inlet portion.
83. Aspect according to any of the previous aspects, wherein:
   the device includes:
      a first fluid inlet portion,
      a second fluid inlet portion,
      a third fluid inlet portion,
   the flow diverter includes:
      a first fluid distribution conduit associated with the first fluid inlet portion and arranged between said at least one fluid supply conduit and one or more outlets of said plurality of fluid admittance outlets,
      a second fluid distribution conduit associated with the second fluid inlet portion and in fluid connection with one or more outlets of said plurality of fluid admittance outlets,
      a third fluid distribution conduit associated with the third fluid inlet portion and in fluid connection with one or more outlets of said plurality of fluid admittance outlets.
84. Aspect according to aspect 82 or 83, in which the flow diverter does not have a fluid outflow conduit.
85. Aspect according to any one of the preceding aspects, in which the device comprises at least two fluid supply lines to supply fluid to the culture chamber, i.e:
   a first fluid supply line having the flow diverter as the fluid admittance organ into the chamber,
   a second fluid supply line configured to admit an additional flow of fluid into the culture chamber.
86. Aspect according to any of the previous aspects, in which the second supply line provides for fluid to be fed into the culture chamber downstream of a pre-chamber.
87. Aspect according to any of the previous aspects, in which the flow diverter is configured to change the flow lines of fluid being admitted into the culture chamber.
88. Aspect according to any of the previous aspects, in which the flow diverter is a predominantly solid component.
89. Aspect according to any of the previous aspects, in which the flow diverter has one or more hollow portions configured to allow fluid to pass through the flow diverter.
90. Aspect according to any of the previous aspects, in which the flow diverter is a predominantly solid component whose cavities are exclusively configured to allow fluid to pass through it.
91. Aspect according to any of the previous aspects, in which the flow diverter is made of biocompatible and sterilisable material.
92. Aspect according to any of the above aspects, in which the flow diverter and/or chamber is produced by 3D printing.
93. Aspect according to any of the preceding aspects, in which the flow diverter is configured to divide a flow entering the cell culture device into a plurality of fluid flows delivered within the culture chamber.
94. Aspect according to any of the previous aspects, in which the flow diverter is a generator of toroidal fluid motion, in particular a generator of turbulent toroidal fluid motion.
95. Aspect according to any of the previous aspects, in which the flow diverter is a flow distributor.
96. Aspect according to any of the previous aspects, in which the flow diverter is a separate component from the culture chamber.
97. Aspect according to any of the previous aspects, in which the culture chamber has a side wall and a lower portion, the side wall and the lower portion being in one piece.
98. Aspect according to any of the previous aspects, in which the side wall of the chamber is in one piece.
99. Aspect according to any of the previous aspects, in which the flow diverter has a basically ‘mushroom-shape’.
100. Aspect according to any of the previous aspects, wherein the flow diverter has a stem in which a supply conduit is defined and a body engaged above the stem, the body being tapered from bottom to top.
101. Aspect according to any of the previous aspects, in which the body of the flow diverter has a substantially ‘bell-shaped’ conformation.
102. Aspect according to any one of the preceding aspects, wherein the device comprises a closure element, the culture chamber being superiorly open and the closure element being configured to superiorly fluid-tight close the culture chamber in an assembly condition in which it is engaged to the culture chamber.
103. Aspect according to any of the previous aspects, in which the chamber has a substantially axisymmetrical conformation.
104. Aspect according to any of the previous aspects, in which the chamber has a substantially cylindrical conformation.
105. Aspect according to any of the preceding aspects, in which the device includes, at one of its lower portions, a second fluid inlet portion.
106. Aspect according to any one of the preceding aspects, wherein the device comprises a pre-chamber arranged, with reference to a fluid advancement direction, downstream of the second fluid inlet portion and upstream of an inner volume of the culture chamber, the flow diverter forming, at the fluid outlet of the pre-chamber, at least one fluid passage port with a bottom portion of the culture chamber.
107. Aspect according to any of the previous aspects, in which the second portion of the fluid inlet is connected to a second inlet in the flow diverter.
108. Aspect according to any of the preceding aspects, in which the device includes, at one of its lower portions, a third fluid inlet portion.
109. Aspect according to any of the previous aspects, in which the third portion of the fluid inlet is connected to a third inlet in the flow diverter.
110. Aspect according to any of the preceding aspects, wherein the device comprises, at one of its upper portions, an opening allowing fluid to be drawn or drained from the chamber.
111. Aspect according to any of the preceding aspects, in which said opening is defined at a closure element of the device.
112. Aspect according to any of the preceding aspects, in which the flow diverter is at least partially accommodated in a defined housing at a lower portion of the device.
113. Aspect according to any of the preceding aspects, in which a stem of the flow diverter within which a fluid supply conduit is defined is engaged in the housing.
114. Aspect according to any of the previous aspects, in which the chamber is elongated, where elongated chamber means a chamber in which the height is greater than the diameter.
115. Aspect according to any of the preceding aspects, in which the chamber has a diameter and a height, the height being greater than the diameter.
116. Aspect according to any of the above aspects, in which the height of the chamber is at least 1.3 times the diameter of the chamber.
117. Use of a cell suspension culture device in accordance with any of the preceding aspects and/or the appended claims to cultivate cells in suspension.
118. Use of a cell suspension culture device in accordance with any of the preceding aspects and/or the appended claims for tests on suspension cultured cells and/or for the production, e.g. on a large scale, of cells for scientific or industrial use and/or for producing vaccines.
119. A method of operating a cell culture device, preferably a cell suspension culture device, in accordance with any one of the preceding aspects and/or the appended claims, the method comprising the following steps:
   admitting fluid into said device through said at least one fluid inlet portion,
   downstream of said at least one fluid inlet portion and upstream of said at least one fluid outlet portion, conveying the fluid from said at least one fluid inlet portion into the flow diverter arranged at least partially within the culture chamber, the upstream and downstream arrangements being defined with reference to a fluid advancement direction,
   admitting, by the flow diverter's plurality of fluid admittance outlets, a plurality of fluid flows into the culture chamber.
120. Aspect according to any of the preceding method aspects, wherein the stop of admitting a plurality of fluid flows into the culture chamber comprises generating a toroidal fluid motion or field in the culture chamber.
121. Aspect according to any of the preceding method aspects, in which the toroidal fluid motion or field in the chamber is at least partially turbulent.
122. Aspect according to any of the previous method aspects, in which the toroidal fluid motion or field in the culture chamber comprises a turbulent component and a laminar component.
123. Aspect according to any of the preceding aspects of the method, wherein the step of admitting a plurality of fluid flows into the culture chamber comprises generating a rotational fluid motion or field in the culture chamber.
124. Aspect according to any of the previous method aspects, in which the rotational fluid motion or field in the culture chamber is turbulent.
125. Aspect according to any one of the preceding aspects of the method, wherein the step of admitting a plurality of fluid flows into the culture chamber comprises generating both a toroidal motion of fluid in the culture chamber and a rotational motion of fluid in the culture chamber.
126. Aspect according to any of the previous method aspects, where the method is a cell suspension culture method.
127. Aspect according to any of the preceding method aspects, in which the fluid is or comprises fluid of or for cell culture.
128. Aspect according to any of the above method aspects, in which the fluid includes cells in suspension.
129. Aspect according to any one of the preceding aspects of the method, wherein the step of admitting a plurality of fluid flows into the culture chamber comprises directing the plurality of fluid flows transversely with respect to a wall, e.g. a bottom wall and/or a side wall of the culture chamber, delimiting the culture chamber.
130. Aspect according to any of the preceding aspects of the method, wherein the step of admitting a plurality of fluid flows into the culture chamber comprises admitting a plurality of turbulent flows into the culture chamber.
131. Aspect according to any one of the preceding aspects of method, wherein the method comprises the step of conveying fluid from said at least one fluid inlet through said flow diverter.
132. Aspect according to any of the previous aspects of the method, in which the method includes the step of introducing an additional fluid flow into the culture chamber.
133. Aspect according to any of the preceding aspects of the method, wherein the step of introducing an additional fluid flow into the culture chamber comprises making fluid flow into a defined fluid passage port defined between the flow diverter and a bottom wall of the culture chamber.
134. Aspect according to any of the preceding aspects of the method, wherein the step of making fluid flow in a fluid passage port defined between the flow diverter and a bottom wall of the culture chamber comprises making fluid flow in an annular fluid passage port defined between a convex edge of the bottom wall of the culture chamber and a bottom wall of the body of the flow diverter.
135. Aspect according to any of the preceding aspects, in which the convex edge is arranged between a concavity of the bottom wall of the flow diverter and the bottom opening.
136. Aspect according to any of the preceding aspects of the method, in which the method involves admitting a plurality of fluid flows into the culture chamber in a given sequence and/or intermittently with respect to each other.
137. Aspect according to any of the previous aspects of the method, in which the method involves feeding each flow into the culture chamber for a specific period of time.
138. Aspect according to any of the previous aspects, in which the culture device is a bioreactor.
139. Aspect according to any of the previous aspects, in which the culture device is a bioreactor for culturing cells in suspension.
140. Aspect according to any of the previous aspects, in which the cell culture device is of the passive type, i.e. does not include any rotating components to create microgravity/agitate the fluid.
141. Aspect according to any of the previous aspects, in which the flow diverter is a substantially fixed component within the culture chamber.
142. Aspect according to any of the previous aspects, in which the flow diverter is configured to accelerate and/or energise a flow entering the culture chamber.
143. Aspect according to any of the previous aspects, in which the flow diverter is a turbulence generator.
144. Aspect according to any of the previous aspects, in which the flow diverter is configured to generate turbulence as the fluid is admitted in the culture chamber.
145. Aspect according to any one of the preceding aspects, wherein each outlet of the plurality of fluid admittance outlets constitutes a fluid passage restriction capable of accelerating fluid exiting the flow diverter.
146. Aspect according to any one of the preceding aspects, wherein each outlet of the plurality of fluid admittance outlets comprises a nozzle.
147. Aspect according to any one of the preceding aspects, wherein each outlet of the plurality of fluid admittance outlets has a fluid outlet cross-section that is smaller than a fluid passage cross-section of the distribution conduit from which it is fed.
148. Aspect according to any of the previous aspects, where the flow diverter complies with the following formula:

$$k = Q / ( n * A * d / 2 )$$

wherein:
   Q is a volumetric flow rate of fluid, particularly at the inlet to the flow diverter, e.g. in cm³/s,
   n is the number of fluid admittance outlets of the flow diverter,
   A is the cross-section of the fluid admittance outlets, e.g. in cm²,
   d is the internal diameter of the culture chamber of the device, e.g. in cm,
   k has the dimensions of an angular frequency expressed in Hz.
149. Aspect according to any of the previous aspects, where k is between 10 and 40, particularly between 20 and 30.
150. Aspect according to any of the previous aspects, where k is between 45 and 75, particularly between 55 and 65.
151. Cell culture device, preferably suspension and/or adhesion cell culture device, comprising:
   a culture chamber,
   at least one fluid inlet portion configured to enable fluid destined to the culture chamber to enter,
   at least one fluid outlet portion configured to enable exit of fluid from the culture chamber.
152. Aspect according to aspect 151, wherein the device comprises a flow diverter arranged at least partially within the chamber and arranged, with reference to a fluid advancement direction, between said at least one fluid inlet portion and said at least one fluid outlet portion.
153. Aspect according to aspect 151 or 152, in which the flow diverter presents:
   at least one fluid inlet in fluid communication with said at least one fluid inlet portion, and
   at least one fluid admittance outlet section or a plurality of fluid admittance outlets in fluid communication with said at least one fluid inlet of the flow diverter.
154. Aspect according to aspect 153, in which the flow diverter is configured to:
   convey fluid into the culture chamber from said at least one fluid inlet portion,
   introduce, through said fluid admittance outlet section or plurality of fluid admittance outlets, a fluid flow or a plurality of fluid flows into the culture chamber.
155. Aspect according to any one of the preceding aspects, wherein the device additionally comprises at least one holder configured to support one or more scaffolds, preferably to support a plurality of scaffolds.
156. Aspect according to aspect 155, in which the flow diverter includes a positioning element configured to position the holder in the culture chamber.
157. Aspect according to aspect 156, wherein the positioning element is defined at a top portion of the flow diverter and/or comprises a strut, such as a central strut, of the flow diverter.
158. Aspect according to aspect 156 or 157, wherein the positioning element is configured to position the holder stably and/or univocally and/or in a determined manner or position within the culture chamber.
159. Aspect according to any of the preceding aspects, in which the flow diverter is built-in in the culture chamber, in particular is built-in in a base and/or a bottom wall and/or a side wall of the culture chamber.
160. Aspect according to any one of aspects 155 to 159, wherein the holder has a plurality of housings, each of which is configured to accommodate a respective scaffold.
161. Aspect according to any of aspects 155 to 160, in which the holder also has an interface portion configured to allow the support to interface with the flow diverter.
162. Aspect according to aspect 161, in which the interface portion is a positioning portion configured to position, preferably centre, the scaffold holder with respect to the flow diverter.
163. Aspect according to aspect 160 or 161 or 162, wherein the interface or positioning portion is defined at a central portion of the holder and the housings of the plurality of housings are defined around the interface or positioning portion.
164. Aspect according to any one of aspects 156 to 163, wherein the positioning element is configured to interface with, in particular to be positioned with respect to, an interface or positioning portion of the holder.
165. Aspect according to aspect 164, in which the positioning element and the interface or positioning portion are of corresponding conformation.
166. Aspect according to aspect 164 or 165, in which the positioning element and the interface or positioning portion are offset from each other.
167. Aspect according to any one of aspects 156 to 166, wherein the interface or positioning portion comprises a housing portion and the positioning element comprises a central strut (preferably defining the top portion of the flow diverter) that is insertable within the housing portion of the holder to determine a mutual constraint.
168. Aspect according to any of the preceding aspects, in which the device additionally comprises one or more scaffolds, preferably a plurality of scaffolds.
169. Aspect according to any one of the preceding aspects and in particular at least according to aspect 167 and aspect 155, wherein the one or more scaffolds are supportable or supported by the scaffold holder.
170. System comprising a plurality of culture devices according to any one of the preceding device aspects and/or the appended device claims.
171. Aspect according to aspect 170, in which the cell culture devices are in parallel with each other.
172. Use of a cell culture device according to any of the preceding device aspects and/or the appended device claims, or a system according to aspects 170 or 171, for tissue engineering.
173. Process of assembling a cell culture device comprising the following steps:
   providing a side wall and a base to form a culture chamber of a cell culture device, preferably the cell culture device being in accordance with any of the preceding device aspects and/or any of the appended device claims,
   providing a flow diverter, optionally where the flow diverter is built-in in the base and therefore this step is part of the step of providing a base,
   providing at least one holder configured to support one or more scaffolds,
   engaging at least one scaffold holder to the said flow diverter.
174. Aspect according to aspect 173, in which:
   the step of providing a side wall and a base to form a culture chamber comprises:
   providing a side wall and base separate from each other, the flow diverter being monolithic with the base,
   engaging the side wall and the base with each other, preferably said step comprising positioning the flow diverter inside the culture chamber,
      optionally, the step of engaging to the flow diverter said at least one scaffold holder is carried out before or after the step of positioning the flow diverter inside the culture chamber.
175. Aspect according to aspect 173 or 174, wherein the step of engaging to said flow diverter at least one scaffold holder involves coupling a central strut of the flow diverter to a housing portion of the scaffold holder.
176. Process of removing at least one scaffold involving the steps of:
providing a device, in accordance with any of the preceding aspects and/or any of the attached device claims, additionally provided with:
   at least one scaffold holder,
   one or more scaffolds engaged to said at least one scaffold holder,
removing a closure element 5 at least partially delimiting the culture chamber 2, preferably by disengaging it from the side wall 4,
removing at least one scaffold from the scaffold holder.
177. Aspect according to aspect 176, wherein the step of removing said at least one scaffold comprises bringing (in particular moving) at least one scaffold-stop from a position in which it constrains said at least one scaffold to a position in which it allows the removal of said at least one scaffold from the scaffold holder.
178. Aspect according to any one of the preceding aspects, wherein the culture device comprises one or more fluid supply conduits, each fluid supply conduit being in the form of a continuous channel and/or having a diameter that is substantially constant or without significant or abrupt change in diameter.
179. Aspect according to any of the above aspects, where the device is of the reusable type or is of the disposable type.
180. Aspect according to any one of aspects 155 to 179, wherein the fluid admittance outlets are configured to admit fluid at or near the scaffold holder, particularly below the holder.

### Conventions and Definitions

Note that in the following detailed description corresponding parts/components/elements are indicated with the same numerical references. The figures may illustrate the subject matter of the invention by means of non-scaled representations; therefore, parts/components/elements illustrated in the accompanying figures and relating to the subject matter of the invention may relate only to schematic representations.

In the context of the present dissertation, the use of terms such as “above”, “superior”, “superiorly”, “below”, “inferior”, “sideways”, “lateral”, “lateral”, “inside”, “internally”, “outside”, “externally”, “horizontal”, “horizontally”, “vertical”, “vertically”, “front”, “frontally”, “rear”, “rearwards”, “right”, “left”, similar terms and variations thereof refer, unless otherwise specifically indicated, to at least one spatial orientation that the object of the invention may assume under conditions of use (see, for example, one or more of the appended figures).

Unless otherwise specifically indicated, the terms “condition” or “configuration” may be used interchangeably in the context of this dissertation. The expression “said at least one” is interchangeable with “each”. The expression “each” and the like do not necessarily imply that there is more than one plurality of elements to which it refers; e.g. “each element” may refer to a single element or to a plurality of elements, depending on the context in which it is used and the embodiment to which it refers.

Unless specifically stated otherwise, expressions such as “upstream”, “downstream” and similar or derived expressions refer to the arrangement of parts/components/elements with respect to the direction of fluid flow along a fluid line or circuit or a particular line or branch of the circuit in which such parts/components/elements are located.

In the context of the present dissertation, one or more of the following definitions/conventions are applicable where appropriate and except where otherwise indicated and/or unless the context excludes it:
“cell suspension culture device” means a device configured to allow the culture of cells in suspension. The cell suspension culture device may also be configured to allow cell culture in adhesion; for this purpose, for example, one or more scaffold holders and optionally one or more scaffolds may be provided. The cell suspension culture device is preferably a bioreactor;
“culture chamber” refers to the chamber designed to allow the culture of cells in the cell culture device in suspension and/or in adhesion. The culture chamber defines an internal volume (culture volume) designed to contain fluid and, indeed, to allow the growth of cells in suspension and/or in adhesion. The culture chamber is typically the main fluid chamber of the suspension and/or adhesion cell culture device. Where the suspension and/or adhesion cell culture device has a plurality of chambers (e.g., a main chamber and a pre-chamber), the culture chamber may be the main fluid chamber, i.e., the chamber of the suspension and/or adhesion cell culture device having the largest volume;
the “internal volume” of the culture chamber is understood to be the internal volume of its main fluid containment portion; in essence, in embodiments in which the culture chamber has several internal volumes, the internal volume is understood to refer to the main volume (any pre-chamber(s) are therefore not part of the internal volume thus understood),
“flow diverter” means a component, housed within the culture chamber or built-in in a portion, preferably a lower portion or base, of the culture chamber, which allows at least one flow entering its body to be conveyed and directed in such a way as to deliver a plurality of fluid flows into the culture chamber. Preferably, the flow diverter also allows the outflow of fluid from the culture chamber;
the expression ‘flow diverter’ is interchangeable with ‘flow distributor’ or ‘turbulence generator’,
“scaffold” (that may also be referred to as “construct”) means one or more artificial structures, usable in tissue engineering (engineering of tissues), configured to house and/or support cell cultures and promote their growth, e.g. until obtaining the regeneration of a damaged tissue. To facilitate the above functions, one or more scaffolds may have nanometric morphological characteristics engineered to emulate the structure of the tissue to be regenerated. For example, in the case of bone scaffolds, the scaffold emulates the bone structure.

The aforesaid conventions and definitions may be used, where necessary, to interpret the claims. If necessary, one or more of said conventions and definitions may be included in one or more of the following claims and/or in one or more of the preceding aspects, in particular when said claims and/or aspects use one or more expressions subject of one or more conventions or definitions.

### BRIEF DESCRIPTION OF THE DRAWINGS

In order to better understand the invention and appreciate its advantages, some of its embodiments are described below, by way of example and not limitation, with reference to the attached figures. In the attached figures, the arrows to which no references are associated indicate the directions of fluid flows under conditions of use of the device and flow diverter or directions of assembly or positioning of components. A brief description of the attached figures follows:

FIG. 1 illustrates a flow diverter in accordance with a first embodiment of the invention, where the outer walls are dashed to better illustrate the defined conduits within the flow diverter, which are illustrated in a continuous line;

FIG. 2 shows a bottom view of the flow diverter of FIG. 1;

FIG. 3 illustrates a frontal section of a cell suspension culture device according to the invention and provided with a culture chamber in the lower portion of which the flow diverter of FIG. 1 is housed;

FIG. 3 also illustrates a detail of a fluid passage port communicating with the culture chamber;

FIG. 4 illustrates a side section of the suspension cell culture device of FIG. 3 within which there is culture fluid (culture medium) and where the toroidal motion generated by the flow passing through the fluid passage port defined between a lower surface of the bottom wall of the flow diverter and an upper surface of the bottom wall of the culture chamber and by the flow exiting the plurality of fluid admittance outlets of the flow diverter is graphically schematised;

FIG. 5 illustrates a flow diverter in accordance with a second embodiment of the invention, where the outer walls are dashed to better illustrate the defined conduits within the flow diverter, which are illustrated in a continuous line;

FIG. 6 shows a side section of a cell suspension culture device according to the invention and equipped with a culture chamber in the lower portion of which the flow diverter of FIG. 5 is housed;

FIG. 7 shows a frontal section of the FIG. 6 device;

FIG. 8 illustrates the section of the device realised according to the sectioning plan VIII-VIII illustrated in FIG. 6. FIG. 8 shows the rotational motion field generated by the exit of fluid from the fluid distribution conduits having helicoidal development;

FIG. 9 illustrates a flow diverter in accordance with a third embodiment of the invention, where the outer walls are dashed to better illustrate the defined conduits within the flow diverter, which are illustrated in a continuous line;

FIG. 10 is a top view of the flow diverter in FIG. 9;

FIG. 11 shows a side section of a cell suspension culture device according to the invention and equipped with a culture chamber in the lower portion of which the flow diverter of FIG. 9 is housed;

FIG. 12 is a frontal section of the device in FIG. 11:

FIG. 13 illustrates a cell suspension culture device in accordance with the invention, equipped with a flow diverter in accordance with the first or second embodiment and connected with an external fluid-dynamic circuit equipped with a pump to allow fluid to move along the circuit to and from the device;

FIG. 14 shows a velocity field map (in technical jargon: streamline: velocity field) illustrating a schematic of a frontal section of one half of the culture chamber (the right half) of the cell suspension culture device in accordance with the invention during cell culture; it shows that, due to the provision of an elongated culture chamber, two vortices are created, one in the lower portion of the culture chamber and one in the upper portion of the culture chamber;

FIG. 15 shows, again with reference to a frontal section of half the chamber as in FIG. 14, a colorimetric velocity map where it is shown that the fluid has higher velocity values at the outlet of the flow diverter (left nozzle representing a fluid admittance outlet of the flow diverter) and at the inlet to the culture chamber (bottom nozzle);

FIG. 16 shows a schematic of the fluid admittance outlet of a flow diverter impacting in a transverse (oblique) direction on a side wall of the chamber;

FIG. 17 shows an exploded view of a cell culture device in accordance with a further embodiment of the invention. In accordance with this embodiment of the invention, the device includes a scaffold holder which, in the assembled condition of the device, is arranged within the culture chamber (see FIG. 23);

FIG. 18 shows a lower portion or base of the device in FIG. 17, in which the flow diverter is built-in;

FIGS. 19A and 19B show two versions of scaffold holder, configured to support circular-based scaffolds (FIG. 19A) and polygonal-based scaffolds (FIG. 19B), respectively;

FIGS. 20A and 20B show steps of positioning the scaffold holder of FIG. 19A (FIG. 20A) and positioning a scaffold against this support (FIG. 20B), respectively;

FIG. 21 shows the step of positioning a scaffold holder, to which the respective scaffolds have previously been committed, at the top portion of the flow diverter;

FIG. 22 shows a section of the device in FIG. 17 in an assembled configuration, from which the scaffold holder and respective scaffolds have been removed (arrows indicate flow directions);

FIG. 23 shows a circuit in which the device in FIG. 17, in an assembled configuration, is connected to a pump configured to circulate fluid in the circuit (the arrows indicate the directions of flow in the circuit and within the culture chamber).

### DETAILED DESCRIPTION OF FORMS OF THE INVENTION

### Cell Suspension Culture Device

A cell suspension culture device in accordance with the invention, which will also be referred to in this text simply as a “device” or “bioreactor”, is generally referred to in the figures by the numerical reference 1. As detailed below, in certain embodiments the culture device 1 may be configured to allow both suspension and adhesion culture.

The cell suspension culture device 1 is passive in that it does not include any rotating components to create microgravity or agitate the fluid.

The device 1 comprises a culture chamber 2 defining an internal volume where, in use, cell culture in suspension takes place.

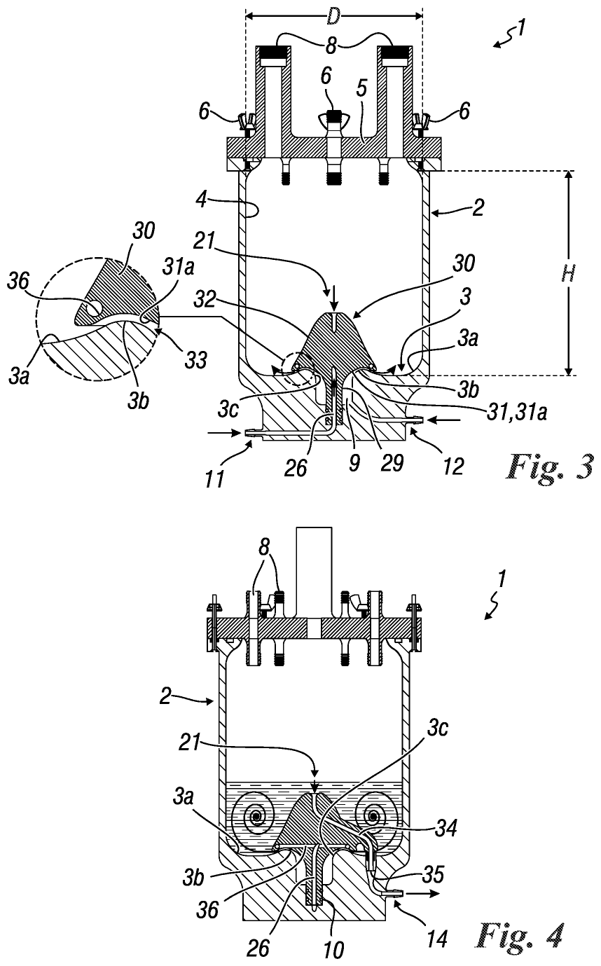

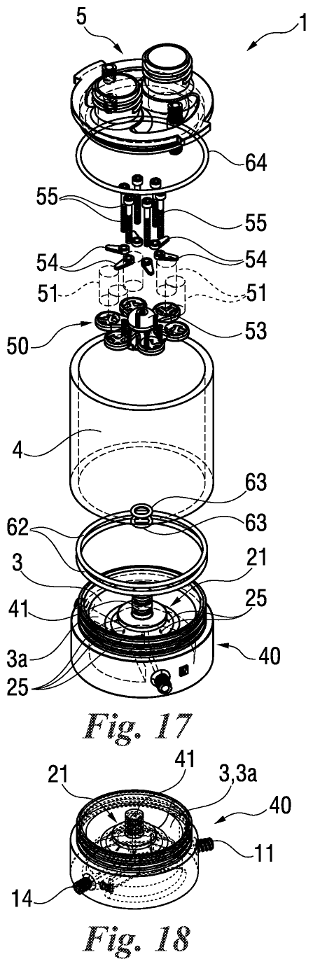

The culture chamber 2 has a bottom wall 3 and a side wall 4 delimiting its internal volume. As illustrated in the attached FIGS. 3, 4, 6, 7, 11, 12, 13, 18 and 21-23, the side wall 4 is preferably in one piece. The provision of a culture chamber 2 delimited laterally by a side wall 4 in a single (monolithic) piece is advantageous compared to embodiments which provide for a side wall of the culture chamber which can be realised by means of two or more components to be assembled together (as in the device of WO2020095143A1) as engagement means (such as threads) are avoided and therefore corresponding fluid leakage or pressure or contamination or inaccuracies or assembly problems that may occur in the assembly of said components are avoided. In addition or alternatively, as illustrated in the appended FIGS. 3, 4, 6, 7 and 11-13, the bottom wall 3 may also be in one piece with the side wall 4; similar reasoning regarding the provision of a monolithic piece and relative advantages is therefore applicable. In another embodiment, illustrated in FIGS. 17, 18, 21, 22 and 23, the bottom wall 3 may be separate with respect to the side wall 4. In such an embodiment, the bottom wall 3 is part of a lower (or base) portion 40 of the culture chamber 2; this lower portion 40 is assembled with the side wall 4. The side wall 4 is superiorly open.

Under conditions of use, the upper opening of the culture chamber 2 is closed by a closure element 5. As illustrated in the appended FIGS. 3, 4, 6, 7, 11, 12, 13 and 17, the closure element 5 is a separate component with respect to the side wall 4 and removable with respect thereto. The closure element 5 is therefore engageable above the side wall 4 in order to close its upper opening. The closure element 5 is engageable to the side wall 4 by means known per se, for example threaded elements 6. The closure element 5 provides a plurality of connections or through-holes 7 or inlets or nozzles 8 for probes for sampling and/or for nutrient insertion and/or for connection to a tank (reservoir) and/or for insertion of an elongated element (such as a tube or rod) for drawing fluid.

Geometrically, and as illustrated in the attached figures, the culture chamber 2 has an axisymmetrical conformation, in particular substantially cylindrical. Preferably, the culture chamber 2 has a substantially constant diameter D. The culture chamber 2 also preferably has a height H greater than the diameter D. Therefore, the culture chamber 2 is elongated; by elongated culture chamber 2 is precisely meant a culture chamber 2 whose height H is greater than its diameter D. If the diameter D is not constant, an average diameter can be taken into account. In more detail, the attached figures show a culture chamber 2 whose height H is at least 1.3 times greater than its diameter D. Providing an elongated culture chamber 2 allows the fluid volume to be varied between various applications/uses, e.g. to allow for an increase in culture fluid when culturing cells for the production of biomolecules. Furthermore, as noted by the fluid dynamic simulations and as visible from FIG. 14, the elongated culture chamber 2 allows for the formation of two separate vortex zones, specifically a lower vortex zone 2′ and an upper vortex zone 2″. Thanks to this field of motion, the cells remain confined in the part below (represented by the lower vortex 2′ which is generated just above the bottom wall 3 of culture chamber 2) and the biomolecules, which have different characteristics, rise in the part above. This solution makes it possible to incorporate both the cells and their products in a single system, thus avoiding the addition of external reservoirs where the collection of biomolecules normally takes place; it is understood that, in some applications, a reservoir can be provided into which the products of the cells, such as antibodies, can be placed. The elongated culture chamber 2 also makes it possible to guarantee, in its upper portion, a reservoir of air sufficient for gaseous exchange, so that it is not necessary to work with the culture chamber filled with fluid. Basically, in use, the device 1 according to the invention operates in the presence, in the culture chamber 2, of culture fluid and also of gas and/or oxygen in the culture chamber (without therefore needing to be filled with culture fluid); in this regard, see FIG. 4. The elongated culture chamber 2 also facilitates the manoeuvres of nutrient insertion, oxygen and CO control₂, pH control as these can be carried out at the upper portion of the device 1, in particular thanks to the connections or through-holes 7 or inlets or nozzles 8 defined in the closure element 5.

The bottom wall 3 of the culture chamber 2 is now structurally detailed. The bottom wall 3 has a concavity 3a and may also have a convex edge 3b and a bottom opening 3c. The convex edge 3b slows the rise of the cells from the concavity 3a and thus allows for improved confinement of the cells in the culture chamber 2 by reducing the risk of deposition of the cells within other cavities below the culture chamber 2; thus, it is possible to operate in the absence of a valve regulating the fluid entry into the culture chamber (such as the valve of the device of WO2020095143A1). The bottom opening 3c is defined at a central portion of the bottom wall 3 and the convex edge 3b is arranged between the concavity 3a and the bottom opening 3c. The convex edge is substantially an edge defining a convexity 3c with respect to the adjacent concavity 3a of the bottom wall 3. The convex edge 3b is therefore elevated with respect to the adjacent concavity 3a; the direction of fluid advancement defined therein runs from the convex edge 3b to the concavity 3a. Both the convex edge 3b and the concavity 3a present an annular development around the bottom opening 3c; in other words, the convex edge 3b and the concavity 3a present a conformation of an annulus developing circumferentially around the bottom opening 3c. Therefore, the provision of a convex edge 3b and a concavity 3a downstream of the convex edge 3b, in particular immediately downstream of the convex edge 3b, advantageously allows for the generation of a toroidal motion of fluid within the culture chamber 2. The toroidal motion allows the movement of fluid (in which the cells are suspended) within the culture chamber 2, which is necessary for cell growth in suspension. The bottom opening 3c preferably has a circular conformation and is arranged in the centre of the bottom wall 3, i.e. is concentric to it.

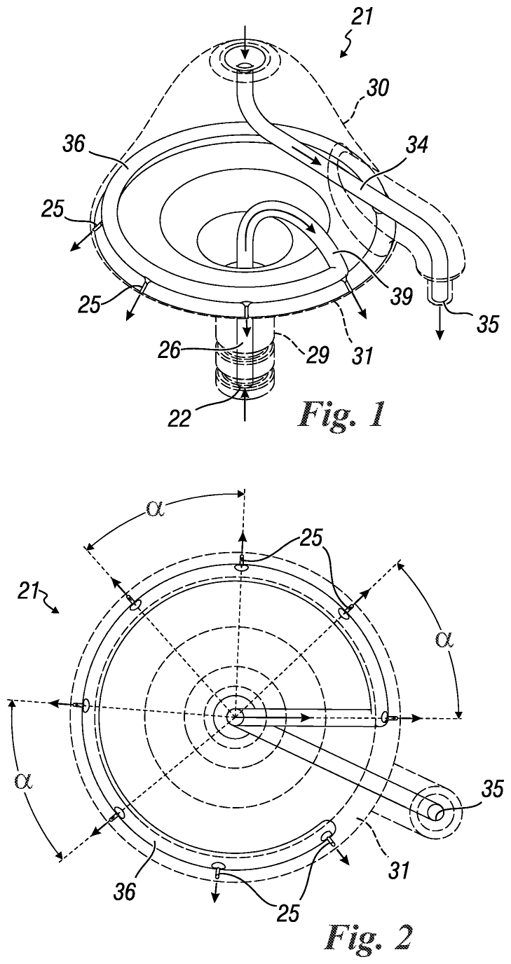

The device 1 may further comprise an additional chamber or pre-chamber 9 defined at a lower portion thereof, in particular below the culture chamber 2; said additional chamber or pre-chamber 9 opens into the bottom opening 3c and is bounded above by it. In the embodiments of FIGS. 1 to 8, the additional chamber or pre-chamber 9 acts as a fluid passage chamber arranged upstream of the culture chamber 2; in essence in accordance with a mode of fluid admittance into the culture chamber 2 the fluid, after passing through the pre-chamber 9, flows into the culture chamber 2. The additional chamber or pre-chamber 9 is a substantially cylindrical well recessed and defined inferiorly with respect to the bottom wall 3. In the embodiment of FIGS. 17 to 23, such a pre-chamber 9 is not provided; however, in some variants of this embodiment, the presence of a pre-chamber is not excluded.

The device 1 may further comprise a housing 10 configured to house at least a portion of a flow diverter 21, which is detailed later in this description. In the attached FIGS. 3, 4, 6, 7, 11 and 12, the housing comprises a hollow portion 10 made at the lower portion of the device 1 and configured to constrain a portion, for example a stem 29, of the flow diverter 21. The hollow portion 10 is communicating with the additional chamber or pre-chamber 9. In the embodiment of FIGS. 17 and 18, the lower portion or base 40 has a built-in flow diverter 21; in other words, the flow diverter 21 is monolithic with the lower portion 40. The flow diverter 21 can therefore be made integral with the lower portion 40; in this way, a monolithic and robust structure is achieved.

With reference to the embodiment of FIGS. 3, 4, 6, 7, 11 and 12, the device 1, in particular the culture chamber 2, is configured to accommodate the flow diverter 21. In greater detail, the device 1, in particular the culture chamber 2, is configured to accommodate the flow diverter 21 at a lower portion of the device 1 itself. As illustrated in the attached FIGS. 3, 4, 6, 7, 11 and 12, the flow diverter 21 may be engaged at (or near) the additional chamber or pre-chamber 9. As illustrated in the attached FIGS. 3, 4, 6, 7, 11 and 12, the stem 29 of the flow diverter 21 is partially housed in the hollow portion 10 which forms the housing communicating with the additional chamber or pre-chamber 9.

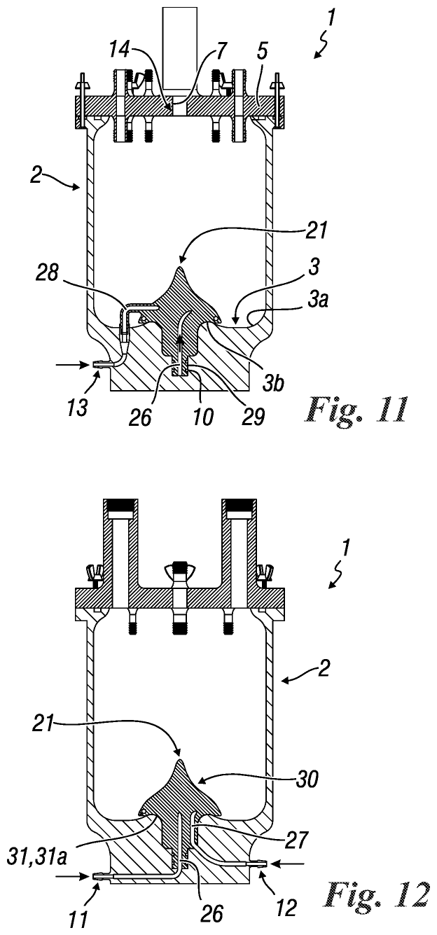

The device 1 further comprises at least one fluid inlet portion 11, 12, 13. The fluid inlet portion 11, 12, 13 allows fluid destined to the culture chamber 2 to enter the device 1. In the appended Figures, the fluid inlet portion 11, 12, 13 is defined at a lower portion of the culture chamber 2 and is in fluid communication with at least one fluid inlet 22, 23, 24 of the flow diverter 21, as will be seen in more detail below. Preferably, the device 1 comprises at least a first and a second fluid inlet portion 11, 12. The first fluid inlet portion 11 is in fluid communication with the flow diverter 21 and the second fluid inlet portion 12 is in fluid communication with the pre-chamber 9 (see FIGS. 3, 4, 6 and 7) or both fluid inlet portions 11, 12 are in fluid communication with the flow diverter 21 (see FIGS. 11 and 12). In the embodiment of FIGS. 11 and 12, the device 1 further comprises a third fluid inlet portion 13 (see FIG. 11), which is also in fluid communication with the flow diverter 21. Each of the fluid inlet portions 11, 12, 13 is preferably defined at the lower portion of the device 1. Each of the fluid inlet portions 11, 12, 13 has, or each of them is associated with, a respective fluid passage channel or conduit or opening, in particular a respective fluid supply conduit 26, 27, 28.

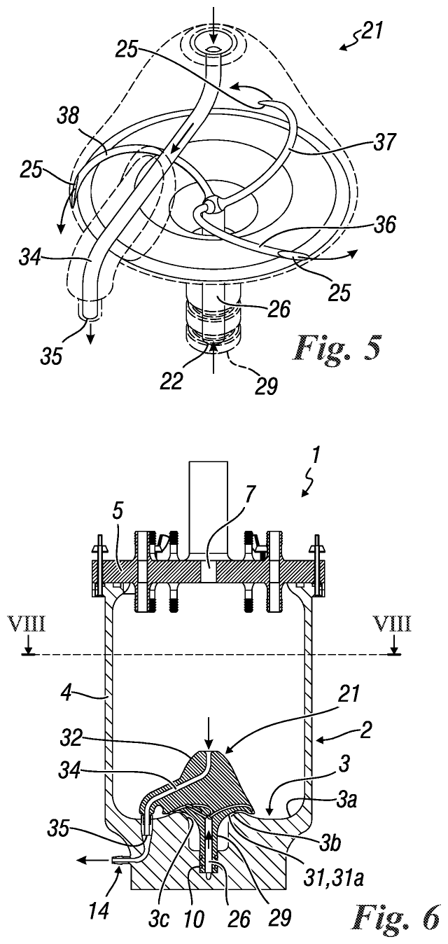

The device 1 further comprises at least one fluid outlet portion 14. The one or more fluid outlet portions 14 allow fluid, originating from the culture chamber 2, to exit the device 1. The fluid outlet portion 14 may be defined at the lower portion of the device 1 and/or at the upper portion of the device 1, in particular at the closure member 5. In the embodiments of FIGS. 3, 4, 6 and 7, the fluid outlet portion 14 is defined at the lower portion of the device 1 and is in fluid communication with the flow diverter 21; optionally, in such embodiments, a through-hole 7 may be provided on the closure element 5 to allow fluid passage for insertion of an elongated element to draw fluid. Normally, under conditions of use of the device 1 of the embodiments of FIGS. 3, 4, 6, 7, 22 and 23, fluid exits the culture chamber 2 via the fluid outlet portion 14 (fluid outlet from below). Differently, in the embodiment of FIGS. 11 and 12, the fluid exit from the culture chamber 2 is provided only at a fluid outlet portion 14 constituted by the through-hole 7, realised on the closure element 5 (see FIG. 11), which allows the insertion of an elongated element to draw fluid (fluid exit from above). In the latter embodiment, no fluid outlet is provided at the lower portion of the device 1; the conduit of the device 1 which is illustrated in FIG. 11 is in fact used to allow fluid to enter a corresponding inlet 24 of the flow diverter 21 (whereas said conduit of the device 1 in the embodiments of FIGS. 4 and 6 is used to allow fluid to exit the device 1). Each fluid outlet portion 14 has, or is associated with, a respective channel or conduit or fluid passage opening.

The device 1 further comprises a flow diverter 21, which can be associated at or near the bottom 3 of the culture chamber 2 or built-in in at least a lower portion 40 of the culture chamber 2.

### Flow Diverter

The flow diverter 21 is described herein with particular reference to the three embodiments illustrated in the attached FIGS. 1-13; each embodiment of the flow diverter 21 corresponds to a respective embodiment of the device 1 comprising said flow diverter 21. It is also contemplated that a flow diverter 21 built-in with the culture chamber 2 may be realised. In use, the flow diverter 21 is arranged within the culture chamber 2 and is engaged at (FIGS. 1-13), or is integral with (FIGS. 17, 28 and 21-23), a lower portion of the culture chamber 2 and of the device 1. When fluid connections are defined below, they refer to the conditions of use of the device 1 and of the flow diverter 21. Before turning to the specifics of each embodiment of the flow diverter 21, features in common between the embodiments are described. Note how the flow diverter 21 is a static component, i.e. it is not configured to rotate within the culture chamber 2. The flow diverter 21 is therefore a substantially fixed component within the culture chamber 2. In terms of material, it is noted that the material of the flow diverter 21 is preferably biocompatible and sterilisable in autoclave; for example, the flow diverter 21 may be made of biocompatible and sterilisable resin. The resin used is preferably configured for use in complex, tough applications and capable of reproducing small parts with a smooth surface finish. The flow diverter 21 (as well as the culture chamber 2) can be produced, preferably in resin, by means of 3D printing technology, in particular 3D stereolithographic printing technology. The flow diverter 21 may be transparent; thus, a visual inspection of the flow diverter 21 may be performed.

The flow diverter 21 comprises at least one fluid inlet 22, 23, 24 in fluid communication with at least one fluid inlet portion 11, 12, 13 of the device 1 so as to receive fluid from the fluid inlet portion 11, 12, 13; in the embodiment in which the flow diverter 21 is integral with the culture chamber 2, the fluid inlet 22 and the fluid inlet portion 11 may coincide or be displaced in immediate proximity to each other, for example with the fluid inlet 22 disposed immediately downstream of the fluid inlet portion 11.

The flow diverter 21 further comprises a plurality of fluid admittance outlets 25, which are in fluid communication with at least one fluid inlet 22, 23, 24 of the flow diverter 21. The fluid admittance outlets 25 are preferably defined on a side and/or bottom surface of the flow diverter 21. The fluid admittance outlets 25 are directed transversely to the bottom wall 3 and/or the side wall 4 of the culture chamber 2 such that the fluid flow is directed towards at least one wall 3, 4 which, due to its concave conformation, directs the fluid flow exiting the flow diverter 21. In particular, the fluid admittance outlets 25 may be oriented obliquely, so that the fluid exiting from them impinges on the bottom wall 3. The angle of inclination of an axis A of the fluid admittance outlets is inclined with respect to both a horizontal direction and a vertical direction (oblique direction of fluid admittance into the culture chamber), and this angle may vary depending on the applications, needs and/or volume of the suspension cell culture device. As depicted in the accompanying figures, each fluid admittance outlet may provide a respective nozzle 25. The fluid admittance outlets 25 are preferably angularly offset from each other; in particular, pairs of fluid admittance outlets adjacent to each other are offset by the same angle α. Providing angularly staggered fluid admittance outlets, in particular by the same angle α, allows a plurality of fluid outflows from the flow diverter 21 to preferably homogeneously cover a wide angular arc, in particular substantially 360° (see attached figures), so that the fluid admittance takes place circumferentially around the flow diverter 21 and a toroidal and/or rotational motion can be generated in the culture chamber 2 all around the flow diverter 21. A homogeneous distribution of all fluid components within the culture chamber 2, including the cells, is a key property to promote nutrient exchange and avoid cell deposition and gradient formation, thereby increasing cell growth. In addition, it is important that the cells are evenly distributed within culture chamber 2 so that each gets the right supply of oxygen and nutrients to perform its metabolic functions.

The flow diverter 21 is configured to accelerate and/or energise an inlet flow to the culture chamber 2; this is achieved by providing a plurality of fluid admittance outlets 25 (nozzles) having a respective fluid passage cross-section that is smaller than the fluid passage cross-section of one or more fluid distribution conduits 36, 37, 38 (described below) to which they are connected.

The flow diverter 21 is structured to convey fluid from at least a portion of the fluid inlets 11, 12, 13 into the culture chamber 2 and to feed, via the plurality of fluid admittance outlets 25, a plurality of fluid flows into the culture chamber 2.

In order to allow fluid inlet, the flow diverter 21 comprises at least one fluid supply conduit 26, 27, 28 which is preferably defined at a lower portion of the flow diverter 21. The at least one supply conduit 26, 27, 28 develops at a central portion of the flow diverter 21.

Structurally, the flow diverter 21 illustrated in the attached FIGS. 1 to 12 comprises a stem 29 and a body 30 connected to the stem 29 and arranged superiorly thereto. The body 30 is in fluid communication with the stem 29. The flow diverter 21 is advantageously monolithic; therefore, the stem 29 and the body 30 are preferably in one piece. The stem 29 may have a generally cylindrical and elongated shape. As illustrated in the appended FIGS. 1 to 12, the stem 29 may develop along a rectilinear direction, in particular along a single rectilinear direction. The fluid supply conduit 26 is defined within the stem 29. In essence, the fluid supply conduit 26 is an internal conduit, i.e. it is formed within a body (or mantle) of the stem 29 of the flow diverter 21. This design choice advantageously allows the flow rates of the device 1 to be as low as possible. As the culture chamber 2 is elongated, this increase in height leads to an increase in the hydrostatic pressure at the base of the device 1, whereby the effect of the cell suspension at the base of the culture chamber 2 decreases. With the configuration of the device 1 in accordance with the invention, there is a “punctual” and localised admittance (i.e. with a plurality of fluid admittance outlets 25 separated from each other) which is able to maintain the same cell suspension effect (the same as would be the case with a non-elongated culture device), ensuring operation at lower, controlled flow rates.

The stem 29 allows not only the fluid inlet to the flow diverter 21, but also the engagement and positioning of the flow diverter 21 with respect to the device 1 and the culture chamber 2. The body 30 has a greater volume than the stem 29 and, in accordance with the illustrated embodiments, has a tapered shape from the bottom upwards so as to minimise the footprint inside the culture chamber 2. The body 30 has a bottom wall 31 and a side wall 32; the side wall 32 is transverse to the bottom wall 31 and is defined by a mantle having substantially revolution symmetry. Preferably, the mantle of the body 30 has a curvilinear profile which allows the flow diverter 21 to minimise its influence on the motion field generated in the culture chamber 2. Preferably, the flow diverter 21 is a solid component whose cavities are provided to allow fluid to pass through it. As illustrated in the attached figures, the body 30 preferably has a substantially “bell-shaped” conformation.

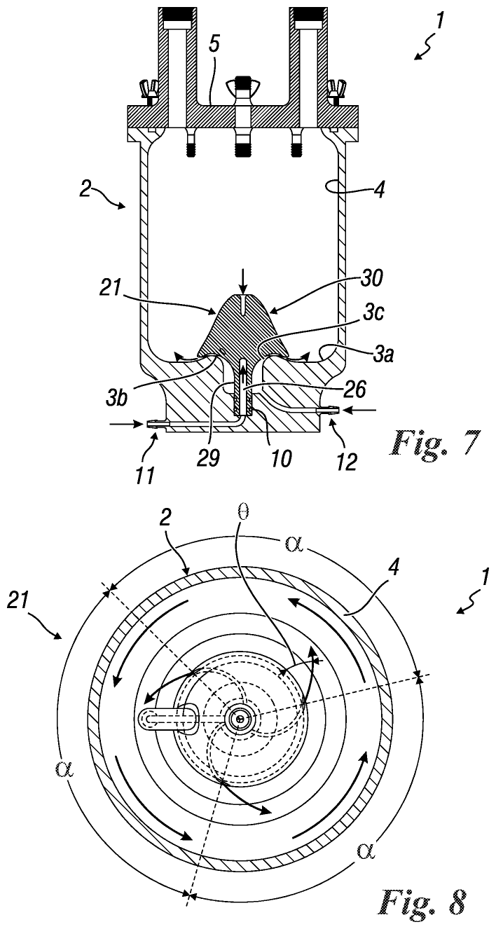

The bottom wall 31 of the body 30 defines a concavity 31a developing annularly around the stem 29. The concavity 31a is defined by a bottom surface of the bottom wall 31. Said concavity 31a corresponds to the convexity of the convex edge 3b annularly developed around the bottom opening 3c of the culture chamber 2. The coupling between the flow diverter 21 and the culture chamber 2 is such that there is a fluid passage port 33 between the concavity 31a of the flow diverter 21 and the convexity of the convex edge 3b (see the detail of FIG. 3 and the arrows indicating the fluid passage in that area in the sections of FIGS. 3 and 7); this is particularly applicable to the first and second embodiments, which provide for a pre-chamber 9 from which fluid flows through the fluid passage port 33. Taking into account the direction of fluid advancement, the presence of a further concavity 3a developing annularly downstream of the convex edge 3b allows the triggering of a toroidal motion field which allows an optimal fluid movement in the culture chamber 2.

As mentioned above, the flow diverter 21 can be engaged and stably positioned relative to the culture chamber 2 of the device 1 at the housing 10. In particular, the stem 29 of the flow diverter 21 is at least partially engaged within the hollow portion defined by the housing 10. As the housing 10 is concentric with the culture chamber 2, such engagement between the stem 29 and the housing 10 of the device 1 allows the flow diverter 21 to be centred with respect to the culture chamber 2.

Some similarities between certain embodiment are described below, and then we move on to the description of the individual embodiments. It should be noted that the development of the three embodiments of flow diverter 21 described herein with reference to FIGS. 1-13 and also of the further embodiment of FIG. 18 derives from a computational and experimental study in which cell suspension was verified and ensured and cell sedimentation on the bottom wall 3 of the culture chamber 2 was avoided; such effects may also derive from further geometries, which may be optimised according to the specific application and/or requirements and/or size (see also the section on scalability).

In the first embodiment (FIGS. 1-4) and in the second embodiment (FIGS. 5-8), the flow diverter 21 is configured to allow, in addition to fluid admittance into the culture chamber 2, fluid outflow from the culture chamber 2. To this end, the flow diverter 21 provides a fluid outflow conduit 34 that develops between a first end and a second end. The first end is an upper end and is defined at an upper portion, such as a top portion, of the flow diverter 21; it is configured to channel, into the fluid outflow conduit, fluid from the culture chamber 2. For the purpose of channeling fluid, it may have a substantially funnel-shaped conformation, which may be defined at an outer surface of the top of the body 30. The second end is a lower end and has at least one fluid outflow outlet 35 suitable for discharging fluid from the culture chamber 2 to the fluid outlet portion 14 of the device 1.

In all three embodiments, the flow diverter 21 comprises at least one fluid distribution conduit 36, 37, 38 at least partially defined in the body 30 and disposed between the fluid supply conduit 26, 27, 28 and one or more outlets of the plurality of fluid admittance outlets 25. The fluid supply conduit 26 is common to all three embodiments and is arranged at a central portion of the flow diverter 21, in particular of its stem 29. The fluid distribution conduit 36, 37, 38 is transversal to the respective fluid supply conduit 26, 27, 28, has an at least partially curvilinear development and is in fluid communication with one or more fluid admittance outlets 25. The fluid distribution conduit 36, 37, 38 has a conformation of an annulus (first embodiment) or a conformation of an annulus portion (third embodiment) or a helicoidal conformation (second embodiment).

The constructive choice of one or more fluid supply conduits 26, 27, 28, in the form of continuous channels having a substantially constant diameter or in any case without significant or abrupt variations in diameter, joining the outside and the inside of the culture chamber 2 derives from the desire not to create cell shear stresses related to the variation of the diameter of the circuit during the passage of the culture fluid from the outside to the inside of the culture chamber 2. This is an improvement with respect to the prior art; indeed, in some bioreactor configurations of the prior art such as WO2020095143A1, this passage takes place in a drastic manner, since the medium coming from the external fluid-dynamic circuit, consisting of tubes, is pushed inside the base provided with a duct having a much larger diameter than that of the external tubes. The above-mentioned construction choice, implemented in the device 1 in accordance with the invention, makes it possible to keep the external and internal circuit (“external” and “internal” are to be interpreted with respect to the culture chamber 2) as homogeneous as possible, without significant variations in diameter, guaranteeing a homogeneous pressure within the entire system, leading the cells to undergo less shear stress. The aforementioned improvements in fluid flow are facilitated by the structure of the flow diverter 21 comprising one or more one fluid supply conduit 26, 27, 28 and in particular by the provision of a stem 29, in which the at least one fluid supply conduit 26, 27, 28 is defined, and a body 30 communicating with the at least one fluid supply conduit 26, 27, 28 of the stem 29.

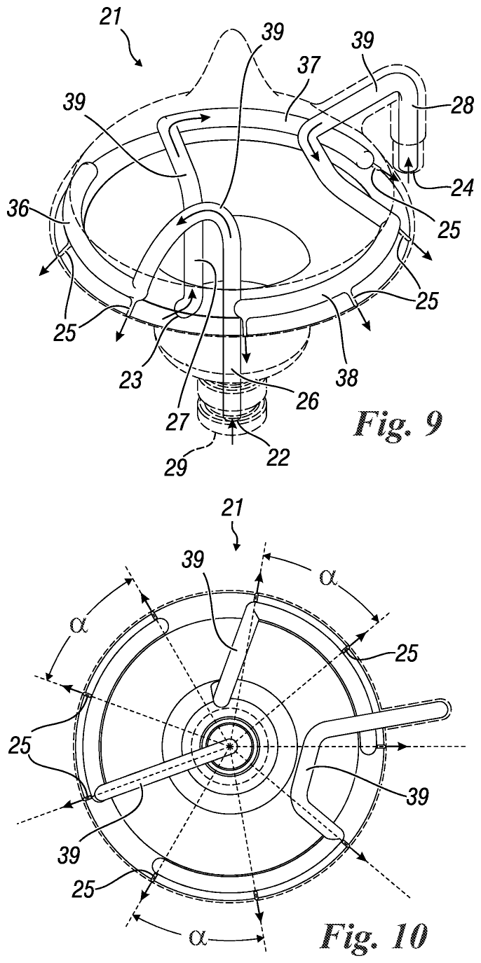

In the first embodiment and in the third embodiment (FIGS. 9-12), in order to connect the fluid supply conduit 26, 27, 28 with the respective fluid distribution conduit 36, 37, 38, the flow diverter 21 may comprise at least one junction conduit 39 disposed between the fluid distribution conduit 36, 37, 38 and the fluid supply conduit 26, 27, 28 and transverse to both. The junction conduit 39 is configured to convey fluid from the fluid supply conduit 26, 27, 28 into the fluid distribution conduit 36, 37, 38.

### First Embodiment (‘Donut’)

The first embodiment, illustrated in FIGS. 1 to 4, envisages a single fluid distribution conduit 36 which develops annularly at a perimeter portion of the body 30 of the flow diverter 21. The single fluid distribution conduit 36 is configured to distribute fluid to all outlets of said plurality of fluid admittance outlets 25. Having such an annular development, the fluid distribution conduit 36 has a development substantially as an annulus or “doughnut-shaped”. As illustrated in FIGS. 1 and 2, the fluid distribution conduit 36 may present an open annular development. This annular development preferably covers an angular arc of at least 270°, in particular of at least 300° (see FIG. 2). Furthermore, by developing annularly, the fluid distribution conduit 36 delimits a free internal space (within the annulus) which allows the passage of one or more other conduits, such as the junction conduit 39. In fact, as illustrated in FIG. 1, at the free internal space the junction conduit 36 may be arranged, which has a curvilinear orientation and connects the fluid supply conduit 26 which develops vertically with the fluid distribution conduit 36 which substantially lies on a horizontal plane. These orientations are defined with reference to the conditions of use of the flow diverter 21, illustrated in FIGS. 3 and 4; it being understood that the development and positioning of the conduits may vary depending on the design choices, the volume available and/or other factors.

The fluid distribution conduit 36 extends in proximity of the bottom wall 31 of the housing 30. Fluid admittance outlets 25 branch off from the fluid distribution conduit 36. In particular, for each fluid admittance outlet 25 there is provided a respective channel branching off and diverging radially from the single fluid distribution conduit 36. As illustrated in FIG. 2, the fluid admittance outlets 25 are angularly staggered by the same angle α (see FIG. 2) and are arranged to substantially cover the entire circumference of the body 30 of the flow diverter 21. In order to allow toroidal motion all around the flow diverter 21, the fluid admittance outlets 25 may be at least four, in particular at least six or seven; FIG. 2 shows eight fluid admittance outlets 25. By covering the fluid distribution conduit 36 an angular arc of at least 270° or at least 300° and providing an appropriate number of fluid admittance outlets 25 angularly staggered along this annular development, it is possible to feed fluid homogeneously into the culture chamber 2. Furthermore, since the outlets are directed towards the bottom wall 3 which has a concavity 3a, they contribute to the toroidal motion which is generated in the culture chamber 2 (see FIG. 2). The fluid admittance from the fluid admittance outlets 25 contributes to the toroidal motion which is generated by the fluid admittance into the culture chamber 2 which occurs in the fluid passage port 33 defined between the flow diverter 21 and the convex edge 3b of the bottom wall 3 of the culture chamber 2. In essence, in accordance with the first embodiment, there are two modes of admittance of fluid into the culture chamber 2: one via the plurality of fluid admittance outlets 25 of the flow diverter 21 and the other via fluid that enters from a second fluid inlet portion 12, passes into the pre-chamber 9 and is channeled into the fluid passage port 33. Both modes of fluid admittance generate a toroidal motion in the culture chamber 2, which is therefore accentuated and enhanced with respect to known cell suspension culture devices (in particular with respect to that described in WO2020095143A1); in greater detail, the toroidal motion exiting the fluid admittance outlets 25 is turbulent, while the toroidal motion exiting the fluid passage port 33 is laminar. The combination of the two toroidal motions turbulent and laminar is optimal to avoid the adhesion of cells to the culture chamber 2 and thus optimise their suspension (embodiments of FIGS. 1-12).

The flow diverter 21 in accordance with the first embodiment further comprises the previously introduced fluid outflow conduit 34, which branches off from a top portion of the body 30, passes internally to the body 30 and emerges laterally from the mantle 32 of the body 30; the fluid outflow outlet 35 is thus defined externally with respect to a main volume of the body 30 and is spaced from the bottom wall 31 of the body 30. This spacing ensures that the toroidal motion field in the culture chamber 2 is not altered. As illustrated in FIGS. 3 and 4, the device 1 in accordance with the first embodiment has, at its lower portion, a first fluid inlet portion 11 and a second fluid inlet portion 12. The first fluid inlet portion 11 is in fluid communication with the fluid supply conduit 26 of the flow diverter 21, and the second fluid inlet portion 12 is in fluid communication with the pre-chamber 9. Also defined at the lower portion of the device 1 is the fluid outlet portion 14 which is in fluid communication with the fluid outflow conduit 34 of the flow diverter 21.

### Second Embodiment (‘Helicoidal’)

The second embodiment, illustrated in FIGS. 5 to 8, has a plurality of fluid distribution conduits 36, 37, 38 branching off from the same fluid supply conduit 26. In essence, in the second embodiment as illustrated in FIG. 5, a junction conduit 39 between fluid supply conduit 26 and fluid distribution conduits 36, 37, 38 is preferably not provided; it is understood that, in some variations, one or more junction conduits may be provided. Each fluid distribution conduit 36, 37, 38 opens into a respective fluid admittance outlet 25; it is understood that in other embodiments it may be contemplated that the same fluid distribution conduit 36, 37, 38 opens into a plurality of fluid admittance outlets 25 and/or that a fluid admittance outlet 25 is in fluid communication with more than one fluid distribution conduit 36, 37, 38.

In essence, in the second embodiment, a plurality of fluid distribution conduits 36, 37, 38 branch “like spokes of a wheel” from the supply conduit 26. Each fluid distribution conduit 36, 37, 38 has an at least partially helicoidal conformation (i.e. defining at least a portion of a helix). The helicoidal development of the fluid distribution conduits 36, 37, 38 makes it possible to generate a rotational flow of fluid in the culture chamber 2 around the flow diverter 21; in this regard, see FIG. 8.

The fluid distribution conduits 36, 37, 38 are configured to feed fluid, by means of respective fluid admittance outlets, at an angle of inclination e between the outlet flow and the base of the body of between 10° to 30°, preferably between 12 and 20°. The inclination angle θ may preferably be about 15°. The inclination angle θ is defined in a horizontal plane; this angle is schematised in FIG. 8.

It is understood that, in other embodiments, the direction of the fluid distribution conduits 36, 37, 38 may be different from the helicoidal direction, provided that it is configured to generate such rotational flow. To this end, the direction of fluid admittance from the admittance outlets 25 of the flow diverter 21 should have at least one motion component tangential to the circumference of the bottom wall 31 of the body 30 and/or have a tilt angle θ as described above.

The fluid admittance outlets 25 are angularly staggered by the same angle α (see FIG. 8) and are arranged to substantially cover the entire circumference of the body 30 of the flow diverter 21. The second embodiment illustrated in FIGS. 5 and 8 has three fluid admittance outlets 25, each of which is served by a respective fluid distribution conduit 36, 37, 38 which are angularly offset from each other by 120°; it is understood that in other embodiments they may be in a number other than three and/or have a different offset angle.

The flow diverter 21 in accordance with the second embodiment also comprises the previously introduced fluid outflow conduit 34 and fluid outflow outlet 35 (see FIG. 5) as previously described for the flow diverter 21 in accordance with the first embodiment. In terms of the fluid inlet and fluid outlet portions, the lower device portion 1 is configured and connected to the flow diverter 21 as in the first embodiment, i.e. with a first and a second fluid inlet portions 11, 12 and with a fluid outlet portion 14.

In essence, also in the second embodiment there are two ways of introducing fluid into the culture chamber 2, one via the plurality of admittance outlets 25 of the flow diverter 21 and the other like that described above, passing through the pre-chamber 9 (flow generating toroidal motion). Note how the admittance of a plurality of flows via the fluid admittance outlets 25 generates in the second embodiment a turbulent rotational motion in the culture chamber 2 (see FIG. 8), whereas in the first embodiment, this inlet generates a toroidal motion; this is due to the different configurations of the flow diverter 21 (different fluid distribution conduits and different arrangement of the fluid admittance outlets). Thus, in the culture chamber 2 a gyroscopic motion is generated by the joint effects (the “sum”) of the toroidal motion and the rotational motion. The Applicant has verified that such gyroscopic motion is particularly effective and suitable for cell culture in suspension. In particular, the turbulent rotational motion suspends the cells and drastically reduces (to the point of eliminating) their deposition on the bottom wall 3 of the culture chamber 2; the combination of rotational and toroidal motion is therefore particularly effective. In addition, it should be noted that the toroidal motion creates a laminar flow within culture chamber 2, ensuring a more homogeneous distribution of the fluid and thus of oxygen and nutrients, and that the turbulent rotational motion, which rotates the cells around the main axis of flow diverter 21, keeps the cells in a convection zone.

### Third Embodiment (‘Tripartite’)

The third embodiment, illustrated in FIGS. 9 to 12, has a plurality of fluid distribution conduits 36, 37, 38. The concept underlying the fluid distribution of this embodiment is to subdivide the single distribution conduit of the first embodiment into a plurality of separate portions 36, 37, 38 (i.e. the following first, second and third distribution conduits), each of which has a conformation of an annulus portion. In the specific embodiment illustrated in detail in FIGS. 9 and 10, the annulus portions 36, 37, 38 are three and therefore the fluid distribution conduit (understood as the sum of the portions) is substantially “tripartite”; it is understood that in other embodiments, a plurality of fluid distribution conduits in a number different from three (multipartite conduit) may be provided.

Turning to the specifics of FIGS. 9 and 10, flow diverter 21 provides:
a first fluid distribution conduit 36 in fluid communication a first fluid inlet portion 11 of the device 1 via the first fluid supply conduit 26,
a second fluid distribution conduit 37 in fluid communication with a second fluid inlet portion 12 of the device 1 via a second fluid supply conduit 27,
a third fluid distribution conduit 38 in fluid communication with a third fluid inlet portion 13 of device 1 via a third fluid supply conduit 28.

As illustrated in FIG. 9, a respective junction conduit 39 may be provided between each fluid supply conduit 26, 27, 28 and each fluid distribution conduit 36, 37, 38. It is understood that, in other embodiments, the number of fluid distribution conduits 36, 37, 38 and/or fluid supply conduits 26, 27, 28 and/or junction conduit 39 may be different from three.

From each fluid distribution conduit 36, 37, 38, channels associated with fluid admittance outlets 25 branch off and diverge radially. Each fluid distribution conduit 36, 37, 38 opens into a respective plurality of fluid admittance outlets 25; in essence, each fluid distribution conduit 36, 37, 38 is associated with a section or group of fluid admittance outlets 25. A fluid admittance section may also comprise only one fluid admittance outlet 25 while a fluid admittance group comprises a plurality of fluid admittance outlets 25. In the specificity of FIGS. 8 and 9, it is contemplated that each fluid distribution conduit 36, 37, 38 opens into a group of fluid admittance outlets comprising three fluid admittance outlets 25. It is understood that in other embodiments, it may be contemplated that the same fluid distribution conduit 36, 37, 38 opens into a group whose fluid admittance outlets 25 are different in number from three (or into a single fluid admittance outlet) and/or that a fluid admittance outlet 25 is in fluid communication with more than one fluid distribution conduit 36, 37, 38.

Each fluid distribution conduit 36, 37, 38 is supplied independently of the others by a respective fluid supply conduit 26, 27, 28. Providing a plurality of fluid distribution conduits 36, 37, 38 is advantageous in that it allows fluid to be supplied to one conduit only or to more than one conduit at a time, for example intermittently and/or sequentially depending on the specifics of the application. In particular, through the use of an automated system, it is possible to choose which fluid distribution conduit(s) 36, 37, 38 (which annulus) to activate by supplying fluid, for how long and in what sequence with respect to each other.

The stem 29 of the flow diverter 21 according to the third embodiment is engaged at the additional chamber or pre-chamber 9 in such a way as to leave no space for the passage of fluid as in the first and second embodiments (see FIGS. 11 and 12); however, consider how a small quantity of fluid (fluid leakage) may enter the additional chamber or pre-chamber 9 and may go to feed one of the three fluid distribution conduits 36, 37, 38. In greater detail, such fluid leakage may pass through the fluid inlet 23 going to feed the fluid distribution conduit 37. In essence, the stem 29 of the flow diverter 30 according to the third embodiment is preferably wider than the stem of the flow diverter of the first and second embodiments.

As illustrated in FIGS. 11 and 12, the device 1 comprises in its lower portion a first, a second and a third fluid inlet portions 11, 12, 13 each of which is in fluid communication, via the respective fluid supply conduit 26, 27, 28, with the first, second and third distribution conduits 36, 37, 38 respectively. The third fluid inlet portion 14 is the portion which in the first and second embodiments serves as the fluid outlet portion; this is possible because the flow diverter 21 in accordance with the third embodiment does not have the possibility of fluid flowing out of the culture chamber 2 via the flow diverter 21 (it is not provided with a fluid outflow conduit nor a fluid outflow outlet). The third fluid supply conduit 28 emerges laterally from the mantle of the body 30; the third fluid inlet 24 is therefore defined externally with respect to a main volume of the body 30 and is spaced from the bottom wall 31 of the body 30. It is understood that in other embodiments, the outflow diverter 21 may also provide a fluid outflow conduit and the device 1 may provide a corresponding fluid outlet portion in fluid communication with the fluid outflow conduit.

From the point of view of the field of motion which the flow diverter 21 with tripartite (or in any case multipartite) distribution conduits generates inside the culture chamber 2, it is indicated that, conceptually deriving its distribution conduits from a fractionation of the single distribution conduit of the first embodiment, the motion generated in the culture chamber is toroidal. For this toroidal motion, which is turbulent by analogy with the first embodiment, the same considerations made above apply; however, in the third embodiment as illustrated in the attached figures, the laminar toroidal motion field deriving from the additional chamber or pre-chamber 9 is not generated since this is substantially full due to the presence of the stem 29.

Possibility of Integrating the Flow Diverter into the Culture Chamber

As anticipated above, in further alternative embodiments to the first, second and third embodiments, the flow diverter 21 may be built-in into the culture chamber 2. In particular, as illustrated in FIGS. 17, 18 and 21-23, the flow diverter 21 may be built-in in the lower portion or base 40 of the culture chamber 2. In such an embodiment, the flow diverter 21 has a body 30 provided with a positioning element 41 (in the appended Figures, in the form of a central strut), which preferably defines a top portion of the body 30.

### Flow Trends, Particularly Downstream of Fluid Admittance Outlets

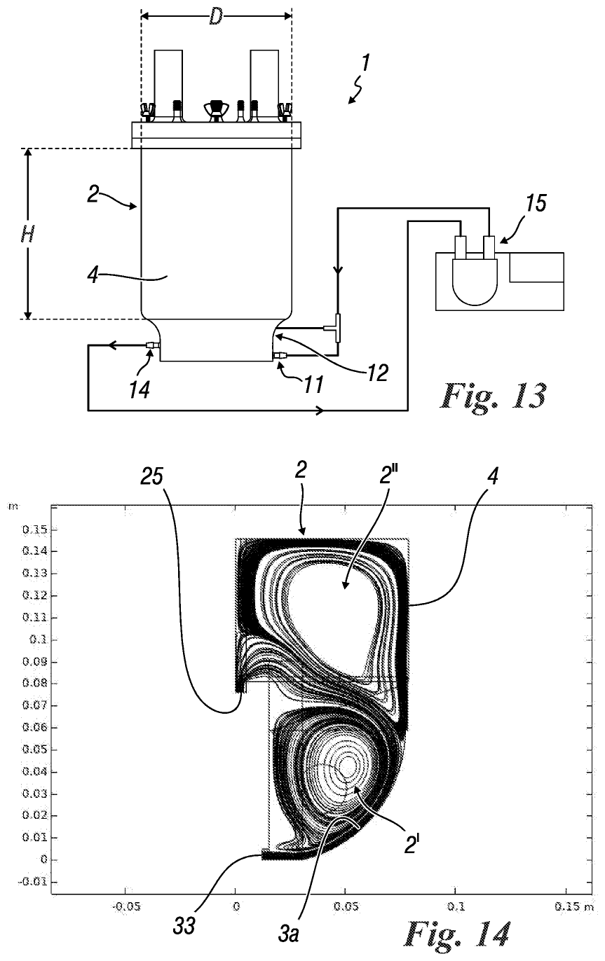

Upstream of device 1, an external fluid-dynamic circuit can be provided, in particular of the closed type, in which there is a pump 15 (peristaltic pump) configured to allow, under operating conditions, the movement of fluid along said circuit (see FIG. 13). In the schematic diagram of FIG. 13, a device 1 comprising a flow diverter 21 according to the first or second embodiment is illustrated; in fact, a fluid outlet portion 14 is shown from which fluid exits (the arrow indicating the fluid direction is exiting from the nozzle or coupling associated with the fluid outlet portion 14). In a schematic (not illustrated in the accompanying figures) in which a device 1 having a flow diverter 21 in accordance with the third embodiment is illustrated, the portion that is illustrated in FIG. 13 as a fluid outlet portion 14 would be a fluid inlet portion 13 (in greater detail the third fluid inlet portion) and the arrow indicating fluid direction would be entering the nozzle or coupling associated with said fluid inlet portion 13. In some applications, a reservoir may be provided into which cell products, such as antibodies, may be fed. By providing a reservoir, the culture chamber 2 remains isolated and protected from the control and monitoring of the parameters required for cell culture carried out in the reservoir. This improves oxygen dissolution through appropriate mechanical agitation imposed in the reservoir and, above all, prevents air bubbles from entering the fluid recirculation system and thus compromising cell viability.

Fluid outflows from the fluid admittance outlet 25 are inclined with respect to the side wall 4 of the culture chamber 2 (see FIG. 8) and impinging on the wall 4 create a rotational motion which is added to the toroidal motion, as described below. In FIG. 16 is schematised a fluid flow exiting a fluid admittance outlet 25 impinging in an oblique direction on the side wall 4 of the culture chamber 2; this fluid flow pattern is applicable to each realised shape, and in particular to the second realised shape. The geometric centre GC is the point defined by the prolongation on the side wall of the axis A of the fluid admittance outlet, the upstream direction is the one facing the acute angle β while the downstream direction is the opposite, in the direction of the main flow. The stagnation point SP is located in the direction of the acute angle β, the maximum pressure point, on the other hand, tends to move away from the stagnation point depending on the inclination of the flow. Leaving the fluid admittance outlet 25 the impacting flow presents a turbulent velocity and kinetic energy profile that varies depending on the set flow rate and the geometry of the flow diverter 21. In the volume located near the fluid admittance outlets 25, the flow is independent of the presence of the side wall 4 of the culture chamber 2, due to the distance between them and the flow diverter 21; the axial velocity, moving away from the fluid admittance outlet, begins to decrease and the flow tends to widen. Due to the impact of the flow on the wall the desired motions are created, rotational in the case where the flow diverter 21 is in accordance with the second embodiment and toroidal in the case where the flow diverter 21 is used in accordance with the first embodiment or the third embodiment. The flow arriving at the wall changes direction and is deflected laterally, generating tangential and normal stresses that influence the local transport of quantities. Here, the flow is influenced by velocity gradients against both the walls and the surrounding fluid. The configuration created increases turbulence and delineates the main flow motion.

Similar considerations regarding fluid flow and fluid dynamics within culture chamber 2 are applicable to the closed circuit of FIG. 23.

Cell Suspension Culture Device with Scaffold Holder

The cell suspension culture device 1 shown in FIG. 17 is equipped with a holder 50 configured to support a plurality of scaffolds 51; this holder 50 is also referred to simply as a ‘scaffold holder’ 50.

The device 1 shown in FIG. 17 is configured for use in tissue engineering applications. This device 1 is scalable and has an operational adaptability that allows it to be used from small-scale to large-scale applications/production.

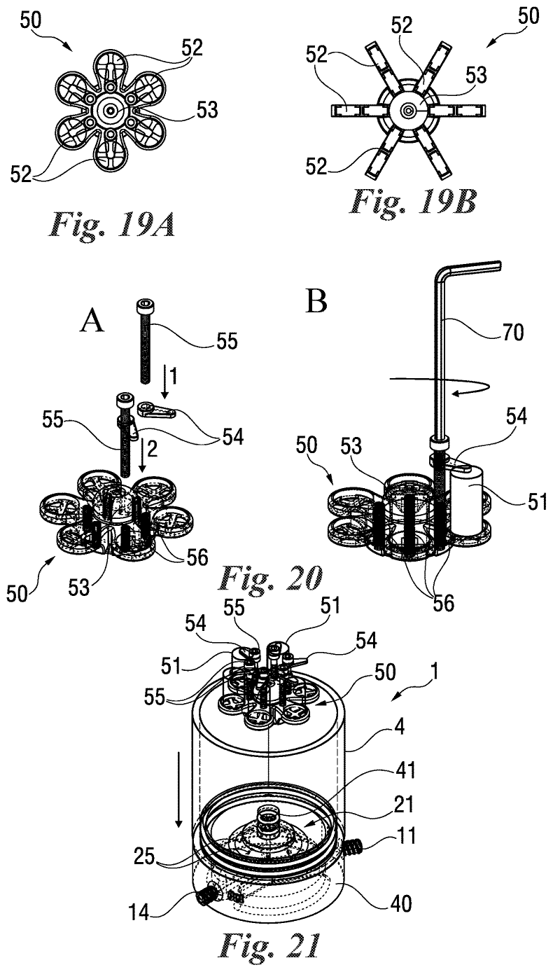

The holder 50 has a plurality of housings 52 each of which is configured to house a respective scaffold 51. The holder 50 further has an interface portion 53 configured to allow the holder 50 to interface with the flow diverter 21. Preferably, the interface portion is a positioning portion 53 configured to allow positioning, particularly centring, of the holder 50 to the flow diverter 21. In the embodiment illustrated in the appended FIGS. 17 to 23 (see in particular FIGS. 19A and 19B), the interface or positioning portion 53 is defined at a central portion of the holder 50 and the housings 52 are defined, for example by a structure like spokes of a wheel, around the interface or positioning portion 53.

FIGS. 19A and 19B show two of the many possible configurations that can be used of the scaffold holder 50; the holder 50 of FIG. 19A is configured to support circular-based scaffolds 51, in particular cylindrical-shaped scaffolds 51 (see FIG. 20), while the holder of FIG. 19B is configured to support polygonal-based scaffolds, in particular parallelepiped-shaped scaffolds 51. FIGS. 19A and 19B show holders 50 that can support up to six scaffolds 51, which may have the same or different dimensions. It is understood that the scaffold holder 50 may be shaped in other ways, to support scaffolds 51 of different geometries and dimensions; the holder 50 may in fact be custom designed and manufactured to support scaffolds 51 of specific geometries. One and the same device 1 may be provided with a plurality of scaffold holders 50, particularly different from each other (such as those in FIGS. 19A and 19B) so as to allow the device 1 to be used with different types of scaffolds 51.

The device 1 equipped with a scaffold holder 50 enables anchorage-dependent (adhesion) cell culture by supporting one or more scaffolds 51 that enable adhesion of cells. Thus, the cell culture device 1 in accordance with the embodiment of FIG. 17, equipped with a scaffold holder 50 per scaffold 51, enables adhesion-dependent cell culture.

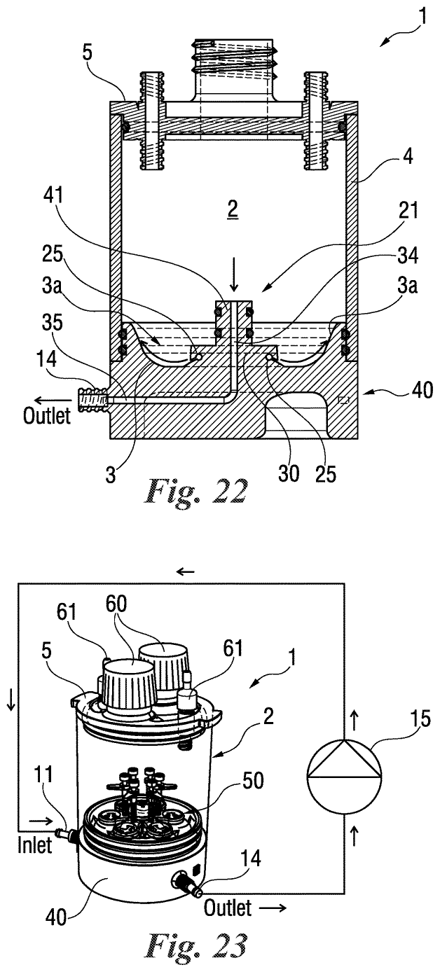

The scaffold holder 50 is engageable to the flow diverter 21. For this purpose, the flow diverter 21 includes a positioning element 41 configured to position the holder in the culture chamber 2. The positioning element 41 is configured to position the holder 50 stably and/or univocally, within the culture chamber 2, relative to the flow diverter 21. Preferably, the positioning element 41 is defined at (or itself defines) a top portion of the flow diverter 21; preferably, the positioning element is defined by a central strut 41 extending into a central portion of the flow diverter 21. The positioning element 41 is configured to interface, in particular to be constrained with, with the interface or positioning portion 53 of the holder 50. Preferably, the positioning element 41 and the interface or positioning portion 53 are of conformation corresponding to each other; in particular, they may be countershaped with each other. Preferably, the positioning portion 53 allows centring the scaffold holder 50 relative to the flow diverter 21 and relative to the culture chamber 2. The interface or positioning portion may be in the form of a housing portion 53 of the holder 50 (FIGS. 19A, 19B) and the positioning element may be in the form of a central strut 41 (FIG. 22) of the flow diverter 21 insertable within the housing portion 53 of the holder 50 to engage with each other. As illustrated in FIGS. 17, 20A and 20B, the housing portion 53 develops in a central portion of the holder 50 and is preferably in the form of a dome, which may be inferiorly hollow to allow the central strut 41 to be housed in the lower cavity.

In the embodiment of FIGS. 17 and 18, the lower portion 40 of the culture chamber 2 has a built-in flow diverter 21 (monolithic with the lower portion 40); therefore, the body 30 of the flow diverter 21 (and the stem 29, if any) is made integral with the lower portion 40. Additionally or alternatively, although FIGS. 17 and 21 show them separately, the lower portion 40 may be built-in with the side wall 4 of the culture chamber 2.

The Applicant has verified that the embodiments of the flow diverter 21 which can preferably be built-in in the lower portion of the culture chamber 2 are the first embodiment (“donut”) and the second embodiment (“helicoidal”); the Applicant has verified that with these embodiments optimal results are achieved in terms of fluid flow and cell culture. However, alternatively, the third embodiment shape of the flow diverter 21 can also be built-in in the lower portion. In the attached FIGS. 17-18 and 21-23, a lower portion is shown in which the built-in flow diverter 21 is in accordance with the first embodiment; some modifications have been made to it, for example to conform the top portion as a positioning element (strut, also built-in with the lower portion of the culture chamber) for the scaffold holder. The features of the flow diverter 21 in accordance with one of the three embodiments previously described with reference to FIGS. 1-12 are applicable mutatis mutandis to the embodiment of FIGS. 17-23; for example, when the flow diverter 21 is built-in into the base 40 and at the same time shares features with the flow diverter in accordance with one of the three embodiments previously described with reference to FIGS. 1-12, the body 30 and the stem 29 of the flow diverter 21 may be viewed as monolithic. With respect to the conduits, fluid inlets and outlets of the flow diverter 21 and base 40, the fluid flow outlet 35 may coincide with, or be defined immediately upstream of, the fluid outlet portion 14 (see FIG. 22) and, similarly, the fluid inlet 22 may coincide with, or be defined immediately downstream of, the fluid inlet portion 11.

The device 1 can also be equipped with:
scaffold-stop elements 54 that allow the scaffolds 51 to be positioned relative to the holder; in FIGS. 17, 20, 21 and 23, the scaffold-stop elements consist of clips 54 equipped with a through-cavity (through-hole),
constraining elements 55 configured to lock the scaffolds against the holder, preferably by cooperating with the scaffold-stop elements; in FIGS. 17, 20, 21 and 12 the constraining elements are threaded (threaded screws),
optionally, a plurality of scaffolds 51; note how device 1 can be supplied without scaffolds as these can be sourced separately.

In order to permit the constraint of the scaffolds 51 to the holder 50, the holder 50 may have constraint portions 56; in the embodiment illustrated in the attached figures, these constraint portions 56 are threaded to permit the attachment of the threaded constraining elements 55.

The steps of constraining the constraining elements and scaffolds to the holder are illustrated in FIGS. 20A and 20B; these steps are part of the assembly process for device 1, which is described below.

We now describe the structure of the culture chamber 2 of the embodiment of device 1 shown in FIG. 17; this structure is modular. The culture chamber 2 comprises:
a lower portion 40 equipped with a flow diverter 21 (in the attached figures, flow diverter 21 is built-in in the lower portion 40; however, in other embodiments, flow diverter 21 can be separated from the lower portion),
a side wall 4 (in the figures it is illustrated as separate from the lower portion 40; however, in other embodiments, it may be integral with the lower portion and/or flow diverter),
a closure element 5 with a plurality of connections or through-holes 7 or inlets or nozzles 8 or sampling ports for cell sampling and threaded supports for housing vent plugs.

To the threaded supports of the closure element 5 can be attached respective filtering screw vent plugs 60 (equipped with a pore membrane) to allow gaseous exchange (O₂, CO₂) and to the sampling ports can be attached connectors 61, e.g. luer lock type connectors, see FIG. 23.

A respective connector 61, e.g. luer lock type connector, can be provided at fluid inlet portions 11, 12, 13 and fluid outlet portion 14.

FIG. 23 shows a closed circuit in which, using an external peristaltic pump 15 and tubing, cell culture medium is continuously pumped into the culture chamber 2 from the base 40 (i.e. at the bottom wall 3, see the curvilinear arrows of both FIGS. 22 and 23), raising the cells and allowing the cells to be seeded onto the scaffolds. In FIG. 23, in order to simplify it, the scaffolds 51 are not shown, which, however, are present under the conditions of use of the device 1 in accordance with the embodiment of FIGS. 17-23. In FIG. 23, note from the curvilinear arrows how fluid exits the fluid admittance outlets 25 below the holder 50, in particular between the bottom wall 3 and the holder 50.

The embodiment in FIGS. 17-23 provides at least the following functionality and benefits:
connection of the culture chamber 2 to the pump circuit 15 in the incubator (where device 1 is located);
sampling of the culture medium during cell culture;
possibility of seeding cells within the culture volume;
possibility of using scaffolds of different sizes;
modularity: more than one device 1 can be used to perform several experiments in parallel. In this case, it is preferable for the peristaltic pump 15 to be equipped with a multi-channel head.

### System

The invention further relates to a system comprising: a plurality of cell culture devices 1 of the type described above; optionally, such cell culture devices 1 are in parallel with each other. The devices 1 of the system can be used to perform experiments, preferably in parallel with each other.

### Scalability of the Cell Suspension Culture Device

Being able to present suspension cell culture devices 1 (bioreactors) and their respective culture chambers 1 significantly varying volumes, in particular from the order of magnitude of millilitres to that of litres or hundreds of litres, the following may also vary: the size, the volume, the number of fluid admittance outlets 25, the number of fluid distribution conduits 36, 37, 38 and other parameters of the flow diverter 21 configured to operate within said devices 1. Thus, it is Applicant's purpose to protect a technical solution regardless of the actual dimensions or parameters (e.g. fluid volumes, base chamber diameters, flow diverter parameters, etc.) of the device 1 and/or the flow diverter 21. In essence, the actual dimensions or parameters of the device 1 and/or the flow diverter 21 may follow one or more “invariant constitutive laws” as the various variables at play in the device 1 change.

This objective, which involves parameterisation, is motivated by the need to make device 1 scalable for use in different contexts, from university laboratories (which use small sizes of 100 millilitres or less) to large pharmaceutical companies (where volumes can range from 10-20 litres to 500-1000 litres).

Therefore, the Applicant has considered the opportunity to use the same construction and operating principle at different sizes, without having to redesign the bioreactor 1 for each individual use. More specifically, considering the flow diverter 21 of the first and third embodiments, the number of fluid admittance outlets 25 from which the flows giving rise to an additional toroidal motion escape depends on the flow rate imposed by the pump 15 and the geometry of the culture chamber 2, in particular its diameter D and the fluid admittance outlet section 25 of the flow diverter 21. Similarly, the same variables can be used in the case of the flow diverter 21 in accordance with the second embodiment where their relationship governs the creation of a rotational motion sufficient to suspend the cells and drastically reduce (to the point of eliminating) their deposition on the bottom wall 3 of the culture chamber 2.

The main parameters considered for the realisation of the model are:
flow rate set by the pump in cm³/s (Q)
number of flow diverter fluid admittance outlets (n)
cross-section of fluid admittance outlets in cm² (A)
internal diameter of the culture chamber of the device in cm (d).

The above four parameters are combined according to the following formula:

$$k = Q / ( n * A * d / 2 )$$

where k has the dimensions of an angular frequency expressed in Hz.

In this way, the optimal configuration for different sizes of the culture chamber 2 is obtained by varying the above parameters so as to keep k between 20 and 30 for the flow diverter 21 according to the first and the third embodiment and between 55 and 65 for the flow diverter 21 according to the second embodiment.

It should be noted that the above formula does not report the density and viscosity values of the medium (culture medium), as it is assumed that bioreactors 1 are always used for cell cultures or tissue engineering, where the culture medium has stable density and viscosity values and can therefore be considered constant in the different configurations.

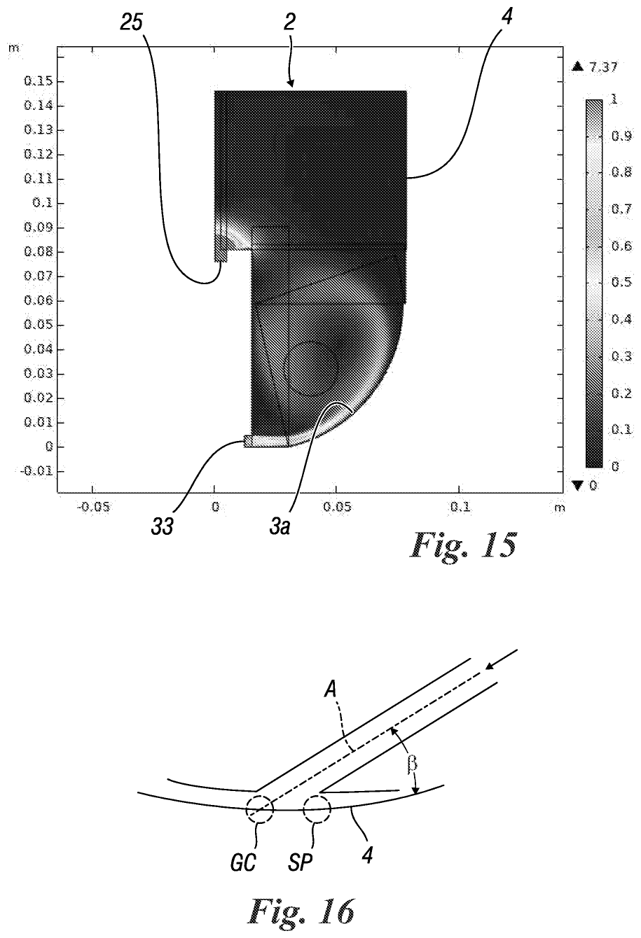

The versatility and adaptability of the device 1 in accordance with the invention thus makes it possible to easily vary the fluid dynamics within the culture chamber 2, in particular by replacing the flow diverter 21 (e.g. with a flow diverter with a different geometry and/or size) depending on the application and requirements and/or either by varying the flow rate, dictated by the pump 15. The flow diverters 21 were designed by means of FEM (Finite Element Method), CFD (Computational Fluid Dynamics) simulations. FIGS. 14 and 15 show 2D simulations in an axisymmetry configuration; note how this is not 100% reflective of the actual flow field generated by flow diverter 21, which has fluid admittance outlets 25 distributed on its side or bottom surface. However, these simulations were used to test the effect of the number of fluid admittance outlets of flow diverter 21, their cross-section and distribution; these aspects were then tested experimentally by means of PIV (Particle Image Velocimetry) analyses and post-processing analyses using appropriate software. The latter two analyses are also used to understand whether gyroscopic motion ensures cell suspension and avoids the sedimentation of cells on the bottom of the culture chamber 21. Note from FIGS. 14 and 15 how the cells remain confined within the bottom vortices (zone 2′) generated in culture chamber 2.

The technical features disclosed herein with respect to functions of device 1 or portions/components/elements thereof are applicable in the context of corresponding uses of device 1 or method steps described below, and may therefore be used to specify such uses and method in the appended claims.

### Use

The invention further relates to a use of the previously described cell suspension culture device 1. The use of the device 1 is directed to cell culture in suspension, optionally both in suspension and in adhesion (see embodiment of FIGS. 17-23).

The device 1 can be used in the laboratory for tests on suspension-cultured cells or for the large-scale production, for example, of cells for scientific or industrial use or in the vaccine production chain.

The use of the device, in particular device 1 equipped with scaffold holder 50, is for tissue engineering operations or applications.

In use, device 1 equipped with a scaffold holder allows both cell suspension culture (particularly at an early stage of its use or deployment) and adhesion cell culture (as the cells adhere to the scaffolds at a later stage) without the need to change culture chamber 2; this is particularly advantageous.

### Method of Operation of a Cell Suspension Device

The present invention also relates to a method of operating a cell culture device 1 in suspension of the type described above. The method comprises the following steps:
introducing fluid into said device 1 through at least one fluid inlet portion 11,
downstream of said at least one fluid inlet portion 11, 12, 13 and upstream of said fluid outlet portion 14, conveying fluid from said at least one fluid inlet portion 11, 12, 13 into the flow diverter 21 arranged at least partially within the culture chamber 2, the upstream and downstream arrangements being defined with reference to a fluid flow direction,
admitting, by means of the plurality of fluid admittance outlets 25 of the flow diverter 21, a plurality of fluid flows into the culture chamber 2.

In accordance with the foregoing, the method may involve generating, by admitting fluid via the plurality of fluid admittance outlets 25, a turbulent toroidal motion of fluid in the culture chamber 2 (first and third embodiments) and/or generating a turbulent rotational motion of fluid in the culture chamber 2 (second embodiment).

In addition, the method may provide, particularly in the first and second embodiments, for the generation of an additional toroidal (laminar) motion in the culture chamber 2 by means of fluid admittance at the fluid passage port 33 downstream of the pre-chamber 9.

The fluid admittance phase may comprise feeding, via the plurality of fluid admittance outlets 25, a plurality of turbulent fluid flows into the culture chamber 2.

The method may also involve, with particular reference to the third embodiment, admitting a plurality of fluid flows into the culture chamber 2 in a given sequence with respect to each other and/or admitting each flow into the culture chamber for a given period of time.

It will be acknowledged by those skilled in the art that, except where otherwise indicated, the particular sequence of steps described herein is purely illustrative and may be varied while remaining within the scope of what is disclosed herein and protected by the appended claims. Thus, except where otherwise indicated or unless specific elements/steps are referred to by use of the same terms, the steps of the method may be carried out in any convenient or desirable order.

Process for Assembling a Cell Suspension Culture Device with Scaffold Holder

The assembly of the device with the scaffold holder (see FIG. 17) is be carried out under sterile conditions. If the same device 1 is used several times, it is to be ensured that any liquids present are removed before sterilisation.

The process includes the assembly phase of scaffold holder 50 and scaffold positioning 51:
engaging a threaded constraining element 55 (hereafter, for simplicity, “screw”) within a constraint portion of a respective scaffold-stop element (hereafter, for simplicity, “clip”) 54, as illustrated in FIG. 20A, see arrow 1. Repeat the operation a number of times equal to the number of scaffolds 51 used;
using a sterile manoeuvring device 70 (e.g. a screwdriver or allen wrench), securing the screw 55 to the scaffold holder 50 at a constraint portion 56 (FIG. 20A, see direction of arrow 2);
using a sterile instrument (e.g. tweezers), placing the scaffold 51 between clip 54 and holder 50 (FIG. 20B);
to secure the scaffold 51, adjusting the height of the screw with the manoeuvring device 70 to allow the clip 54 to secure the scaffold 51 (FIG. 20B); note how the screw has been inserted into a recess in the clip 54. If necessary, rotate clip 54 with the sterile instrument.

It is understood that the above steps of assembling the scaffold holder 50 and the step of positioning the scaffolds 51 may be carried out by alternative means, depending on the type of means adopted to secure the scaffolds 51 to the holder 50.

The process also includes the step of assembling the lower portion (hereinafter, ‘base’) 40 of device 1 to achieve the configuration of FIG. 18. Starting from the exploded view of FIG. 17 (see lower portion of the exploded view), this step involves:
insert two o-rings 62 into the appropriate grooves on the outer surface of the base 40,
insert two o-rings 63 into the appropriate grooves on the central strut 41 of the flow diverter 21 built-in in the base 40;
tighten a luer lock type connector 61 to each fluid inlet portion 11, 12, 13 (side inlet nozzles) and each fluid outlet portion 14 (side outlet nozzle) on the base 40 of the device 1.

The process also includes the step of assembling closure element 5 (hereinafter, ‘cap’), which involves:
inserting an o-ring 64 into the dedicated groove on the outer surface of the closure element 5;
screwing a luer lock type connector 61 onto each threaded port at the top of closure element 5 (see FIG. 17 and then FIG. 23, illustrating the assembled condition where two luer lock type connectors are engaged at the cap);
screwing a vent plug 60 onto each threaded holder on plug 5 (see FIG. 17 and then FIG. 23, illustrating the assembled condition where two vent plugs are engaged to the plug).

If cell sampling is required, tubes and valves can be connected to the luer lock 61 connectors for cell sampling/medium supply.

The process involves assembling side wall 4 to the base and assembling scaffold holder 50 to base 40; note how the order of these steps can be reversed. Furthermore, these steps may vary depending on the nature of the components; for example, if the side wall 4 is integral with the base 40, the step of assembling the side wall 4 to the base is not necessary.

In the construction form shown in FIG. 17, in which side wall 4 and base 40 are separated from each other (the side wall is substantially a hollow cylinder portion), the assembly phase of side wall 4 to the base preferably involves:
moistening the surface of the o-rings 62 of base 40 with water to lubricate them,
coupling the side wall onto the base by applying pressure until the surfaces of the two components make contact (note how the base and side wall have similar diameters).

The assembly of the scaffold holder to the base (see FIG. 21) involves inserting the scaffold support, on which the scaffolds were previously fixed as described above, onto the base by exerting pressure so as to insert the central strut 41 of the flow diverter 21 into the cavity of the housing portion 53 of the scaffold holder 50 and thus constrain the holder to the flow diverter 21.

The process may further comprise connecting an inlet/outlet to the peristaltic pump 15 and introducing a cell suspension/cell medium into the culture chamber 2. The cell medium and cell suspension can be placed directly into the previously assembled base 40 and side wall 4. It is to be ensured that the culture chamber 2 is filled to at least the level of the outlet nozzle.

The process is to mount the closure element 5 on the side wall 4, preferably as follows:
moistening the surface of the o-ring 64 of the closure element 5 with water to lubricate it, so that it is easier to couple closure element 5 and side wall 4,
coupling closure element 5 to side wall 4 by applying pressure until the surfaces of the two components make contact.

In general, the order of the steps in the device assembly process can be changed as required.

At this point, device 1 can be put into operation. Providing the device involves:
switching on the peristaltic pump 15 and set the desired flow rate,
checking for air bubbles in the circuit; due to the presence of air, air bubbles may appear in the tubes and inside the culture chamber 2 in the first few seconds. If the device is working properly, the bubbles will soon dissolve,
switching off the pump 15.

Now proceed to place the device 1 in an incubator. If the peristaltic pump 15 used cannot be placed inside an incubator, ensure that the pump power cable and tubing pass easily through the door seal of the cell culture incubator without compromising the internal environment of the incubator.

The peristaltic pump 15 is then switched on again. If anchorage-dependent (adhesion) cells are used, cell adhesion usually depends on the test conditions.

Cell/medium cell sampling can be performed by connecting a sterile syringe or pipette to the sampling port while device 1 remains inside the incubator or by moving it to a laminar flow hood. This can be performed while the peristaltic pump 15 is running. Alternatively, sampling may be performed by moving the device 1 from the incubator to a laminar flow hood and removing the closure element 5 of the device 1. In this way, a sample can be collected with a pipette or syringe.

Process for Removing at Least One Scaffold from a Cell Suspension Culture Device with Scaffold Holder

The invention further provides a process for removing at least one scaffold 51. This process takes place after the deployment or use of at least one device 1 equipped with a scaffold holder 50 of the type described above and also one or more scaffolds 51. If anchorage-dependent cells are used, adhesion of the cells to the scaffolds 51 can be completed within 24 hours after inoculation of the cell culture. At this point, the cellularised scaffolds 51 (i.e., the scaffolds 51 to whose walls the cells have adhered) can be removed from device 1 to perform physicochemical or biological characterisation. The scaffold holder 50 allows a plurality of scaffolds 51 to be supported to perform statistical and time-dependent analysis of the cellularised scaffolds. To remove scaffolds 51, the device 1 is to be moved from the incubator to a laminar flow hood. The steps for removing at least one scaffold follow:
moving the device 1 from the incubator to a laminar flow hood,
preferably turning off the peristaltic pump 15;
removing the closure element 5, preferably by manually disengaging it from the side wall 4,
using a sterile instrument, such as tweezers, gently rotate the desired scaffold-stop (clip), and
removing the respective scaffold 51.

The removal process may subsequently provide for:
closing the device 1 by gently pushing the closure element 5 onto the side wall 4,
switching on the peristaltic pump 15,
moving the device 1 from the laminar flow hood into the incubator.

### Further Advantages and Concluding Remarks

The provision of the flow diverter 21 advantageously allows the flow to be directed only to certain points in the lower portion of the bioreactor 1. This is an improvement over the prior art, for example WO2020095143A1 in which there was a greater demand and consumption of energy and power from the peristaltic pump; this is because the inlet pressure was dispersed homogeneously over the entire base of the culture chamber. By acting the bioreactor 1 in accordance with the invention in a “punctual” and localised manner, it is possible to decrease fluid flow rates, while maintaining the same cell suspension effect, containing energy consumption and flow rate to controlled values.

The provision of a flow diverter 21 in proximity of or integral with the bottom wall 3 makes the flow diverter 21 ‘built-in’ or integral with the bottom portion of bioreactor 1, making it more compact, easily assembled and sterilised.

The device 1 may be reusable, by sterilisation, or be of the disposable type. The device 1 is preferably configured to be sterilisable in autoclave, for example at a temperature of about 121° C. for about 30 minutes. With regard to the materials, the base, the closure element 5, the scaffold holder 50 and the clips 54 may be made of resin, while the side wall 4 may be made of glass (thus transparent). The screws 55, if provided, may be made of stainless steel. The culture chamber 2 is preferably completely transparent to allow optical investigations.

It has been shown how the invention advantageously enables the provision of a device and method for cell culture in suspension 1 capable of creating an efficient field of motion for cell growth in suspension and, in the embodiment shown in FIGS. 17-23, also in adhesion. In particular, the invention makes it possible to reduce cell adhesion on the wall of the culture device 1, to ensure cell suspension, in the embodiment shown in FIGS. 17-23 also adhesion on the scaffolds 51, to avoid cell sedimentation on the bottom, to more easily control the shear stresses experienced by the cells so that they can be modified as desired according to the type of cells cultured, to increase the homogenisation of nutrients and oxygen and to avoid the formation of gradients of concentration within the culture volume.

It is understood that each element, component and/or phase of the product/method according to the invention may be substituted with an equivalent element, component and/or phase (hereinafter, “equivalent(s)”); such equivalent(s) may already exist on the filing or priority date of this patent text or subsequent conception/development.

1. Cell suspension culture device comprising:
a culture chamber,
at least one fluid inlet portion configured to enable fluid destined to the culture chamber to enter,
at least one fluid outlet portion configured to enable exit of fluid from the culture chamber,
a flow diverter arranged at least partially inside the culture chamber and arranged, with reference to a fluid advancement direction, between said at least one fluid inlet portion and said at least one fluid outlet portion,
the flow diverter having:
at least one fluid inlet fluidically communicating with said at least one fluid inlet portion, and
a plurality of fluid admittance outlets fluidically communicating with said at least one fluid inlet of the flow diverter,
the flow diverter being configured to:
convey into the culture chamber fluid from said at least one fluid inlet portion,
admit, through said plurality of fluid admittance outlets, a plurality of fluid flows into the culture chamber,
wherein each outlet of the plurality of fluid admittance outlets is directed transversally to a wall of the culture chamber, particularly transversally to a bottom wall and/or a lateral wall of the culture chamber,
wherein:
the flow diverter comprises:
at least one stem provided with a fluid supply conduit, and
a body fluidically communicating with said fluid supply conduit, said fluid supply conduit being fluidically communicating, particularly being in direct fluid communication, with said at least one fluid inlet portion and being configured to convey a fluid flow to the body,
the body develops inside the culture chamber and has a volume greater than the stem,
the body comprises a perimetral portion, said plurality of fluid admittance openings being defined at the perimetral portion,
wherein the fluid supply conduit is an inner conduit, preferably wherein it is realised inside a body of a stem of the flow diverter.

2. Device according to claim 1, wherein the perimetral portion is a circumferential portion of the body, the outlets of the plurality of fluid admittance outlets are angularly offset from each other.

3. Device according to claim 1, wherein the flow diverter has a double function:
conveying fluid from said at least one fluid inlet portion into the culture chamber so as to divide it, in particular equally divide it, into a plurality of fluid flows that are fed, via said plurality of fluid admittance outlets, into the culture chamber,
let fluid coming from the culture chamber flow outside the culture chamber.

4. Device according to claim 1, wherein:
the flow diverter further comprises at least one fluid outflow outlet adapted to enable the fluid from the culture chamber to outflow from the culture chamber,
the fluid outflow outlet is in communication with said at least one fluid outlet portion (14),
the flow diverter is configured to convey fluid from said culture chamber, through said fluid outflow outlet, towards said at least one fluid outlet portion thus evacuating fluid from the device.

5. Device according to claim 4, wherein:
the flow diverter is arranged at a lower portion of the culture chamber and said at least one outlet portion is arranged at a lower portion of the cell suspension culture device, and
the flow diverter comprises a fluid outflow conduit which establishes fluid communication between said at least one fluid outflow outlet and said at least one fluid outlet portion,
the fluid outflow conduit is formed at least partially inside the body.

6. Device according to claim 2, wherein the flow diverter comprises at least one fluid distribution conduit defined in the body and arranged between said at least one fluid supply conduit and one or more outlets of said plurality of fluid admittance outlets,
said at least one fluid distribution conduit being transversal to said at least one fluid supply conduit, having an at least partially curvilinear development and being in fluid communication with one or more outlets of said plurality of fluid admittance outlets.

7. Device according to claim 6, wherein:
said at least one fluid supply conduit is arranged at a central portion of the flow diverter,
said at least one fluid distribution conduit has a conformation an annulus or of a portion of annulus,
the flow diverter comprises at least on junction conduit arranged between said at least one fluid distribution conduit and said at least one fluid supply conduit, the junction conduit being configured to convey into said at least one fluid distribution conduit the fluid from said at least one fluid supply conduit.

8. Device according to claim 7, wherein the flow diverter comprises a single fluid distribution conduit having a conformation of an annulus, defined in the body and arranged between said at least one fluid supply conduit and said plurality of fluid admittance outlets,
the single fluid distribution conduit being configured to distribute fluid to all the outlets of said plurality of fluid admittance outlets,
preferably the flow diverter comprising a single fluid inlet fluidically communicating with said single fluid distribution conduit.

9. Device according to claim 7, wherein:
the flow diverter comprises:
a plurality of fluid inlets,
a plurality of fluid distribution conduits each of which has a conformation of a portion of annulus and fluidically communicates with a respective fluid inlet,
in the plurality of fluid admittance outlets, a plurality of fluid admittance sections or groups can be identified, each fluid admittance section or group comprising at least one fluid admittance outlet and being fluidically communicating with a respective fluid distribution conduit,
preferably wherein each fluid distribution conduit fluidically communicates with a fluid admittance group comprising a plurality of fluid admittance outlets.

10. Device according to claim 6, wherein:
said at least one fluid supply conduit is arranged at a central portion of the flow diverter,
the flow diverter comprises a plurality of fluid distribution conduits branching from the fluid supply conduit towards a perimetral portion of the body of the flow diverter,
each fluid distribution conduit has an at least partially helicoidal development,
preferably the flow diverter comprises a single fluid supply conduit and the conduits of the plurality of fluid distribution conduits radially branch from the single fluid supply conduit.

11. Device according to claim 1, wherein:
the device comprises an additional chamber or pre-chamber arranged upstream the culture chamber,
the flow diverter is at least partially housed in said additional chamber or pre-chamber,
preferably said at least one fluid supply conduit develops at least partially at the additional chamber or pre-chamber.

12. Device according to claim 11, wherein:
the culture chamber comprises a bottom wall provided with a bottom opening and with a convex edge developing around the bottom opening,
the pre-chamber is above delimited by said bottom opening and is arranged, with reference to a fluid advancement direction, between at least one fluid inlet portion of the device and an internal volume of the culture chamber,
the flow diverter defines, at the fluid outlet of the pre-chamber, a fluid passage port with the convex edge of the bottom wall of the culture chamber,
preferably wherein the bottom wall of the culture chamber comprises a concavity developing around the convex edge.

13. Device according to claim 1, wherein the culture chamber has a diameter and a height, the height being greater than the diameter, preferably the height being at least 1.3 times the diameter.

14. Device according to claim 1, further comprising at least one holder configured to support one or more scaffolds, preferably to support a plurality of scaffolds,
the flow diverter comprising a positioning element configured to position the holder in the culture chamber.

15. Device according to claim 14, wherein the positioning element is defined at a top portion of the flow diverter, preferably the positioning element comprising a strut of the flow diverter.

16. Device according to claim 14, wherein the positioning element is configured to position the holder stably and/or univocally.

17. Device according to claim 1, wherein the flow diverter is built-in in the culture chamber, in particular is built-in in a base and/or in a bottom wall and/or in a side wall of the culture chamber.

18. Use of the device according to claim 1, for tissue engineering.

19. Method of operating a cell suspension culture device according to claim 1, the method comprising the following steps:
admitting fluid into said device through said at least one fluid inlet portion,
downstream said at least one fluid inlet portion and upstream said at least one fluid outlet portion, conveying the fluid from said at least one fluid inlet portion into the flow diverter arranged at least partially inside the culture chamber, the upstream and downstream arrangements being defined with reference to a fluid advancement direction,
admitting, by the plurality of fluid admittance outlets of the flow diverter, a plurality of fluid flows into the culture chamber.

20. Method according to claim 19, wherein said step of admitting a plurality of fluid flows into the culture chamber comprises:
generating a toroidal flow motion in the culture chamber, and/or
generating a rotational fluid motion in the culture chamber.

21.-25. (canceled)
